(19) United States
(12) Patent Application Publication
(10) Pub. No.: US 2026/0315217 A1
(43) Pub. Date: Oct. 15, 2026

(54) REINFORCEMENT POSTS SECURED TO LEAFLETS OF PROSTHETIC VALVES

(71) Applicant: Edwards Lifesciences Corporation, Irvine, CA (US)

(72) Inventors: Elena Sherman, Pardes Hana (IL); Bich Hoang Pham, Irvine, CA (US); Roy Shitrit, Zichron Yaakov (IL); Brendan Michael Dalbow, Oceanside, CA (US); Svetlana Morduchovitch, Be'er Ya'akov (IL); Tamir S. Levi, Zikhron Yaakov (IL)

(73) Assignee: Edwards Lifesciences Corporation, Irvine, CA (US)

(21) Appl. No.: 19/682,306

(22) Filed: May 19, 2026

Related U.S. Application Data
(63) Continuation of application No. PCT/US2024/057226, filed on Nov. 25, 2024.
(60) Provisional application No. 63/604,291, filed on Nov. 30, 2023.

(51) Int. Cl. A61F 2/24

(52) U.S. Cl. CPC A61F 2/2415; A61F 2/2418; A61F 2220/0075; A61F 2240/001

(57) ABSTRACT

The present disclosure relates to prosthetic valves and method for forming commissures thereof. In an example, a prosthetic valve can include a commissures secured to commissure supports of a frame. Each commissure can include two tabs of adjacent leaflets of the prosthetic valve, with a reinforcement post secured to each tab by a securement stitching pattern which comprises a post securement vertical stitch line positioned radially outwards from the commissure support. In some examples, the securement stitching pattern can be formed off the frame.

### CROSS-REFERENCE TO RELATED APPLICATIONS

This application is a continuation of International Application No. PCT/US2024/057226, filed Nov. 25, 2024, which claims the benefit of U.S. Provisional Application No. 63/604,291, filed Nov. 30, 2023, which is incorporated by reference herein.

### FIELD

The present disclosure relates to prosthetic valves, and in particular, to commissures formed by paired leaflets having reinforcement posts secured thereto.

### BACKGROUND

Native heart valves, such as the aortic, pulmonary and mitral valves, function to assure adequate directional flow from and to the heart, and between the heart's chambers, to supply blood to the whole cardiovascular system. Various valvular diseases can render the valves ineffective and require replacement with artificial valves. Surgical procedures can be performed to repair or replace a heart valve. Surgeries are prone to an abundance of clinical complications, hence alternative less invasive techniques of delivering a prosthetic heart valve over a catheter and implanting it over the native malfunctioning valve, have been developed over the years.

Different types of prosthetic heart valves are known to date, including balloon expandable valve, self-expandable valves and mechanically-expandable valves. Different methods of delivery and implantation are also known, and may vary according to the site of implantation and the type of prosthetic valve. One exemplary technique includes utilization of a delivery assembly for delivering a prosthetic valve in a crimped state, from an incision which can be located at the patient's femoral or iliac artery, towards the native malfunctioning valve. Once the prosthetic valve is properly positioned at the desired site of implantation, it can be expanded against the surrounding anatomy, such as an annulus of a native valve, and the delivery assembly can be retrieved thereafter.

The prosthetic heart valve can include valvular structure (formed by two or more leaflets) for regulating blood flow in a single direction. The leaflets can form commissures between adjacent ones of the leaflets. The commissures can be attached to a frame of the prosthetic heart valve.

### SUMMARY

The present disclosure is directed towards prosthetic valves comprising leaflets attached thereto via commissures that include paired tabs of the leaflets passed through window openings of commissure supports of a frame of the prosthetic valve, wherein the commissure includes reinforcement posts secured to the tabs by securement stitching patterns arranged to simplify the assembly procedure.

According to some aspects of the disclosure, there is provided a method for forming a commissure of a prosthetic valve, the method comprising attaching a reinforcement post to a tab of each leaflet of a plurality of leaflets, by threading a suture through the reinforcement post and the tab along a post securement vertical stitch line, wherein the post securement vertical stitch line does not extend along an entire height of the tab.

In some examples, the method further comprises extending two tabs, one from each of two adjacent leaflets of the plurality of leaflets, through a window opening of a commissure support of a frame of a prosthetic valve, such that the post securement vertical stitch lines are positioned radially outwards from the commissure support.

In some examples, the method further comprises attaching the two tabs of the two leaflets to sidewalls of the commissure support.

In some examples, the method further comprises, prior to the extending the two tabs through the window opening, forming a first folded portion of the reinforcement post by folding an inner edge of the reinforcement post outwards.

In some examples, the method further comprises threading the suture through the first folded portion along an inner fold vertical stitch line.

In some examples, the extending the two tabs through the window opening further comprises positioning the inner fold vertical stitch line radially inwards to the commissure support.

In some examples, the method further comprises, prior to the extending the two tabs through the window opening, forming at least one horizontal stitch extending between the inner fold vertical stitch line and the post securement vertical stitch line

In some examples, the inner fold vertical stitch line, the post securement vertical stitch line, and the at least one horizontal stitch, are continuously formed by a single suture.

In some examples, the post securement vertical stitch line comprises two vertical stitches.

In some examples, the two vertical stitches of the post securement vertical stitch line comprise an uppermost vertical stitch which is distally offset from an outflow edge of the tab, and a lowermost vertical stitch which is proximally offset from an inflow edge of the tab.

In some examples, the uppermost vertical stitch of the post securement vertical stitch line is disposed over a first side of the reinforcement post, and wherein the lowermost vertical stitch of the post securement vertical stitch line is disposed over an inner surface of the tab.

In some examples, a tail of the suture extends from an uppermost penetration point of the uppermost vertical stitch of the post securement vertical stitch line, at least partially along the inner surface of the tab.

In some examples, the inner fold vertical stitch line comprises two vertical stitches.

In some examples, the two vertical stitches of the inner fold vertical stitch line comprise an uppermost vertical stitch which is distally offset from an outflow edge of the reinforcement post, and a lowermost vertical stitch which is proximally offset from an inflow edge of the reinforcement post.

In some examples, the uppermost vertical stitch of the inner fold vertical stitch line is disposed over an outer surface of the first folded portion, and wherein the lowermost vertical stitch of the inner fold vertical stitch line is disposed over an inner surface of the first folded portion.

In some examples, the method further comprises, after the extending the two tabs through the window opening, forming a second folded portion of the reinforcement post positioned radially outwards to the commissure support.

In some examples, the method further comprises threading the suture through the second folded portion along an outer fold vertical stitch line.

In some examples, the outer fold vertical stitch line comprises an uppermost loop extending around an outflow edge of the reinforcement post, and a lowermost loop extending around an inflow edge of the reinforcement post.

In some examples, the outer fold vertical stitch line further comprises an uppermost vertical stitch which is distal to the uppermost loop, and two vertical stitches extending between the uppermost vertical stitch and the lowermost loop, wherein the two vertical stitches extend between mutual penetration points, each over an opposite surface of the second folded portion.

According to some aspects of the disclosure, there is provided method for forming a commissure of a prosthetic valve, the method comprising, for each reinforcement post of a plurality of reinforcement posts, forming a first folded portion of the reinforcement post by folding an inner edge of the reinforcement post.

In some examples, the method further comprises threading a suture through the first folded portion along an inner fold vertical stitch line.

In some examples, the method further comprises attaching one of the plurality of reinforcement posts to a tab of each leaflet of a plurality of leaflets, by forming a horizontal stitch and threading the suture through the reinforcement post and the tab along a post securement vertical stitch line, wherein the horizontal stitch extends between the inner fold vertical stitch line and the post securement vertical stitch line.

In some examples, the method further comprises extending two tabs, one from each of two adjacent leaflets of the plurality of leaflets, through a window opening of a commissure support of a frame of a prosthetic valve, such that the post securement vertical stitch lines are positioned radially outwards from the commissure support, and such that the inner fold vertical stitch lines are positioned radially inwards to the commissure support.

In some examples, the method further comprises attaching the two tabs of the two leaflets to sidewalls of the commissure support.

In some examples, the inner fold vertical stitch line does not extend through the leaflet.

According to some aspects of the disclosure, there is provided a prosthetic valve comprising: a frame, a plurality of leaflets, and a plurality of commissures. The frame comprises a plurality of commissure supports. Each commissure support comprises two sidewalls defining a window opening therebetween. Each leaflet comprises opposing tabs disposed on opposite sides of the leaflet. The plurality of commissure are secured to the plurality of commissure supports.

Each commissure comprises two tabs, one from each of two adjacent leaflets of the plurality of leaflets, and two reinforcement posts, each one of the reinforcement posts attached to a corresponding one of the two tabs of the commissure by a securement stitching pattern which comprises a post securement vertical stitch line positioned radially outwards from the commissure support.

In some examples, the post securement vertical stitch line does not extend along an entire height of the tab.

In some examples, the securement stitching pattern is formed off the frame.

In some examples, each securement stitching pattern is formed by a single suture.

In some examples, each reinforcement post comprises a first folded portion, and the securement stitching pattern further comprises an inner fold vertical stitch line radially inwards to the commissure support, extending through the first folded portion.

In some examples, each securement stitching pattern further comprises at least one horizontal stitch extending between the inner fold vertical stitch line and the post securement vertical stitch line.

In some examples, the inner fold vertical stitch line does not extend through the leaflet.

The aspects of this disclosure can be used in combination or separately. This summary is provided to introduce a selection of concepts in a simplified form that are further described below in the detailed description. This summary is not intended to identify key features or essential features of the claimed subject matter, nor is it intended to be used to limit the scope of the claimed subject matter. The foregoing and other objects, features, and advantages of the invention will become more apparent from the following detailed description, which proceeds with reference to the accompanying figures.

### BRIEF DESCRIPTION OF THE FIGURES

Some examples of the invention are described herein with reference to the accompanying figures. The description, together with the figures, makes apparent to a person having ordinary skill in the art how some examples may be practiced. The figures are for the purpose of illustrative description and no attempt is made to show structural details of an example in more detail than is necessary for a fundamental understanding of the invention. For the sake of clarity, some objects depicted in the figures are not to scale.

In the Figures:

FIG. 1A is a perspective side view of an exemplary prosthetic valve.

FIG. 1B is a perspective view of an annular frame of the prosthetic valve of FIG. 1A.

FIG. 2A is a front view of an exemplary leaflet.

FIG. 2B shows an exemplary valvular structure that includes a plurality of leaflets.

FIG. 3 shows an exemplary delivery apparatus carrying an exemplary prosthetic valve.

FIG. 4 is a front view of a leaflet with attached reinforcement posts according to an example.

FIGS. 5A-5C are sectioned top plan views at various stages of assembly within one of the commissure supports of the frame for leaflets with attached reinforcement posts as shown in FIG. 4.

FIG. 6A is a front view of an exemplary reinforcement post.

FIG. 6B shows an exemplary vertical stitch line formed through a first folded portion of a reinforcement post.

FIG. 6C shows an exemplary tab of a leaflet.

FIG. 6D shows an exemplary stitching pattern comprising the vertical stitch line of FIG. 6B.

FIGS. 7A-7B show an exemplary securement stitching pattern from different view angles.

FIG. 7C shows tabs with reinforcement post having the securement stitching pattern of FIGS. 7A-7B, passed through a window opening of a commissure support of the frame.

FIG. 7D shows the tabs of FIG. 7C spread away from each other.

FIGS. 8A-8B show another exemplary securement stitching pattern from different view angles.

FIG. 8C shows tabs with reinforcement post having the securement stitching pattern of FIGS. 8A-8B, passed through a window opening of a commissure support of the frame.

FIGS. 9A-9B show another exemplary securement stitching pattern from different view angles.

FIGS. 10A-10F show exemplary stages in a method for forming a commissure.

FIGS. 11A-11B show another exemplary securement stitching pattern from different view angles.

FIG. 11C shows tabs with reinforcement post having the securement stitching pattern of FIGS. 11A-11B, passed through a window opening of a commissure support of the frame.

FIG. 11D shows the tabs of FIG. 11C spread away from each other.

FIG. 11E shows a complementary stitching pattern following the securement stitching pattern shown in FIGS. 11A-11D.

FIG. 12A shows another exemplary securement stitching pattern.

FIG. 12B shows a complementary stitching pattern following the securement stitching pattern of FIG. 12A.

FIG. 13 shows another exemplary securement stitching pattern.

FIGS. 14A-14B show another exemplary securement stitching pattern from different view angles.

FIGS. 15A-15B show another exemplary securement stitching pattern from different view angles.

FIG. 16A is a front view of an exemplary reinforcement post that includes notches.

FIG. 16B shows an exemplary leaflet tab devoid of pre-formed apertures.

FIG. 17A shows an exemplary reinforcement post having a marking aperture, laid over a corresponding tab of a leaflet.

FIG. 17B shows another exemplary securement stitching pattern.

### DETAILED DESCRIPTION

For purposes of this description, certain aspects, advantages, and novel features of the examples of this disclosure are described herein. The disclosed methods, apparatus, and systems should not be construed as being limiting in any way. Instead, the present disclosure is directed toward all novel and nonobvious features and aspects of the various disclosed examples, alone and in various combinations and sub-combinations with one another. The methods, apparatus, and systems are not limited to any specific aspect or feature or combination thereof, nor do the disclosed examples require that any one or more specific advantages be present, or problems be solved. The technologies from any example can be combined with the technologies described in any one or more of the other examples. In view of the many possible examples to which the principles of the disclosed technology may be applied, it should be recognized that the illustrated examples are only preferred examples and should not be taken as limiting the scope of the disclosed technology.

Although the operations of some of the disclosed examples are described in a particular, sequential order for convenient presentation, it should be understood that this manner of description encompasses rearrangement, unless a particular ordering is required by specific language set forth below. For example, operations described sequentially may in some cases be rearranged or performed concurrently. Moreover, for the sake of simplicity, the attached figures may not show the various ways in which the disclosed methods can be used in conjunction with other methods. Additionally, the description sometimes uses terms like “provide” or “achieve” to describe the disclosed methods. These terms are high-level abstractions of the actual operations that are performed. The actual operations that correspond to these terms may vary depending on the particular implementation and are readily discernible by one of ordinary skill in the art.

All features described herein are independent of one another and, except where structurally impossible, can be used in combination with any other feature described herein.

As used in this application and in the claims, the singular forms “a,” “an,” and “the” include the plural forms unless the context clearly dictates otherwise. Additionally, the terms “have” or “includes” means “comprises”. Further, the terms “coupled”, “connected”, and “attached”, as used herein, are interchangeable and generally mean physically, mechanically, chemically, magnetically, and/or electrically coupled or linked and does not exclude the presence of intermediate elements between the coupled or associated items absent specific contrary language. As used herein, “and/or” means “and” or “or”, as well as “and” and “or”.

Directions and other relative references may be used to facilitate discussion of the drawings and principles herein, but are not intended to be limiting. For example, certain terms may be used such as “inner,” “outer,” “upper,” “lower,” “inside,” “outside,”, “top,” “bottom,” “interior,” “exterior,” “left,” right,” and the like. Such terms are used, where applicable, to provide some clarity of description when dealing with relative relationships, particularly with respect to the illustrated examples. Such terms are not, however, intended to imply absolute relationships, positions, and/or orientations. For example, with respect to an object, an “upper” part can become a “lower” part simply by turning the object over. Nevertheless, it is still the same part and the object remains the same.

The term “plurality” or “plural” when used together with an element means two or more of the element. Directions and other relative references (e.g., inner and outer, upper and lower, above and below, left and right, and proximal and distal) may be used to facilitate discussion of the drawings and principles herein but are not intended to be limiting.

The terms “proximal” and “distal” are defined relative to the use position of a delivery apparatus. In general, the end of the delivery apparatus closest to the user of the apparatus is the proximal end, and the end of the delivery apparatus farthest from the user (e.g., the end that is inserted into a patient's body) is the distal end. The term “proximal” when used with two spatially separated positions or parts of an object can be understood to mean closer to or oriented towards the proximal end of the delivery apparatus. The term “distal” when used with two spatially separated positions or parts of an object can be understood to mean closer to or oriented towards the distal end of the delivery apparatus. The terms “longitudinal” and “axial” are interchangeable, and refer to an axis extending in the proximal and distal directions, unless otherwise expressly defined.

Throughout the figures of the drawings, different superscripts for the same reference numerals are used to denote different examples of the same elements. Examples of the disclosed devices and systems may include any combination of different examples of the same elements. Specifically, any reference to an element without a superscript may refer to any alternative example of the same element denoted with a superscript. In order to avoid undue clutter from having too many reference numbers and lead lines on a particular drawing, some components will be introduced via one or more drawings and not explicitly identified in every subsequent drawing that contains that component.

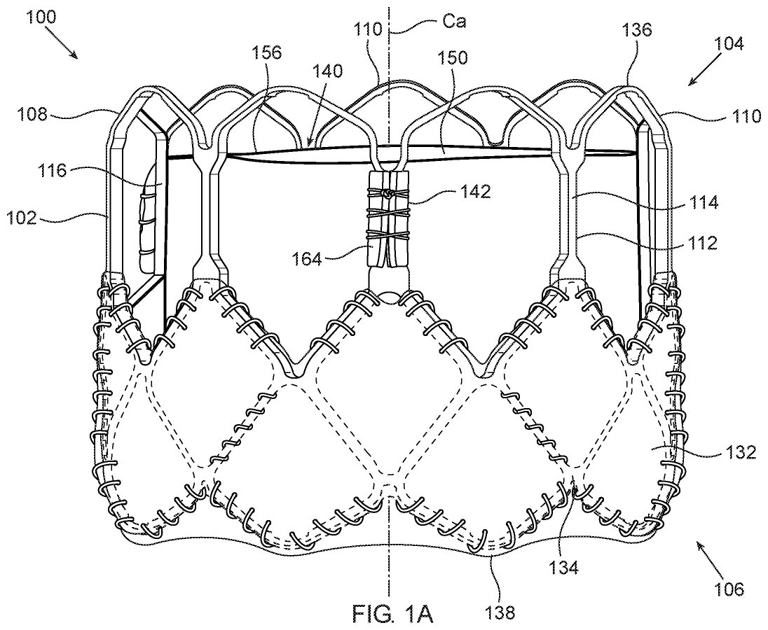

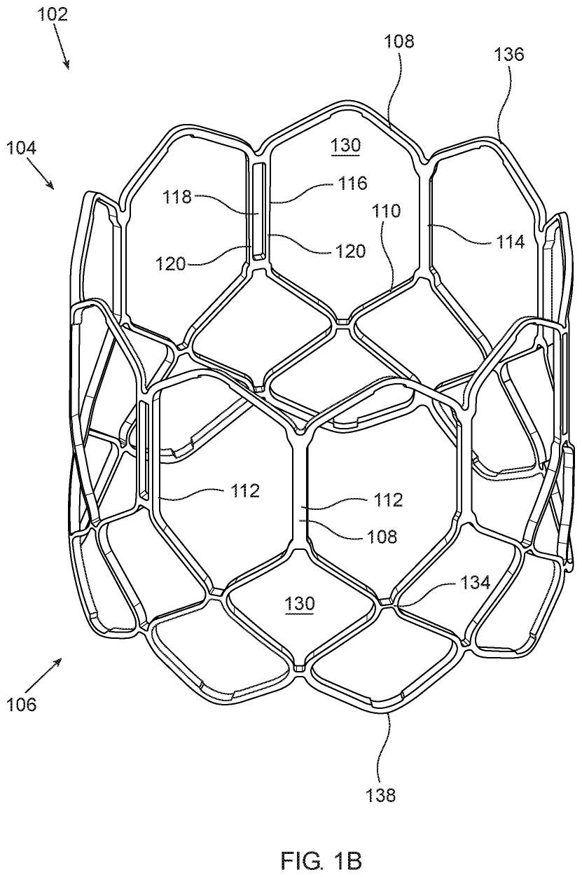

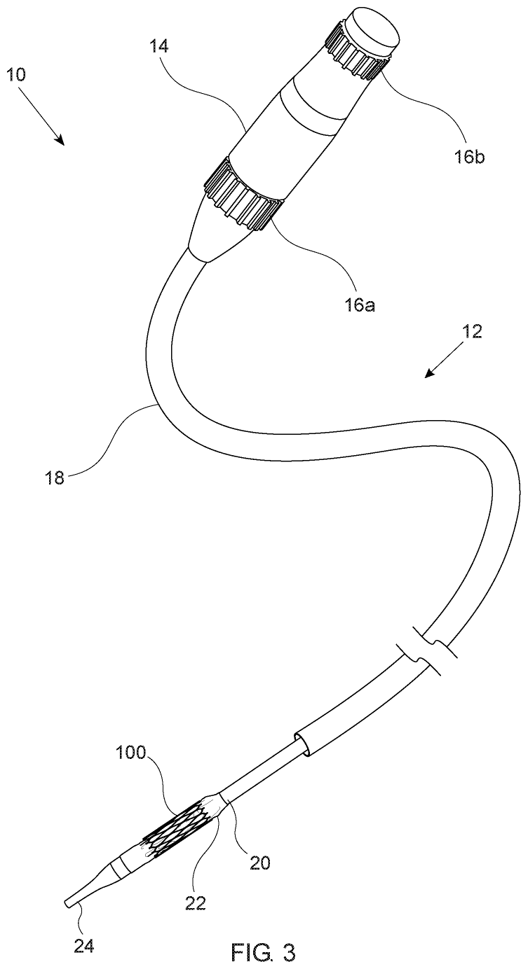

FIGS. 1A and 1B show perspective views of an example of a prosthetic valve 100, with and without soft components (such as skirts and a leaflet assembly), respectively. The term “prosthetic valve”, as used herein, refers to any type of a prosthetic valve deliverable to a patient's target site over a catheter, which is radially expandable and compressible between a radially compressed, or crimped, state, and a radially expanded state. Thus, the prosthetic valves can be crimped on or retained by an implant delivery apparatus 12 (see FIG. 3) in the radially compressed state during delivery, and then expanded to the radially expanded state once the prosthetic valve reaches the implantation site. The expanded state may include a range of diameters to which the valve may expand, between the compressed state and a maximal diameter reached at a fully expanded state. Thus, a plurality of partially expanded states may relate to any expansion diameter between radially compressed or crimped state, and maximally expanded state. A prosthetic valve 100 of the current disclosure may include any prosthetic valve configured to be mounted within the native aortic valve, the native mitral valve, the native pulmonary valve, and the native tricuspid valve.

It is to be understood that the prosthetic valves disclosed herein may be used with a variety of implant delivery apparatuses. Balloon expandable valves generally involve a procedure of inflating a balloon within a prosthetic valve, thereby expanding the prosthetic valve within the desired implantation site. Once the valve is sufficiently expanded, the balloon is deflated and retrieved along with a delivery apparatus 12 (see FIG. 3). Self-expandable valves include a frame that is shape-set to automatically expand as soon an outer retaining shaft or capsule (not shown) is withdrawn proximally relative to the prosthetic valve. Mechanically expandable valves are a category of prosthetic valves that rely on a mechanical actuation mechanism for expansion. The mechanical actuation mechanism usually includes a plurality of expansion and locking assemblies (such as the prosthetic valves described in U.S. Pat. No. 10,603,165, International Application No. PCT/US2021/052745, and U.S. Provisional Application Nos. 63/085,947 and 63/209904, each of which is incorporated herein by reference in its entirety), releasably coupled to respective actuation assemblies of a delivery apparatus, controlled via a handle (not shown) for actuating the expansion and locking assemblies to expand the prosthetic valve to a desired diameter. The expansion and locking assemblies may optionally lock the valve's diameter to prevent undesired recompression thereof, and disconnection of the actuation assemblies from the expansion and locking assemblies, to enable retrieval of the delivery apparatus once the prosthetic valve is properly positioned at the desired site of implantation.

The term “plurality”, as used herein, means more than one.

The prosthetic valve 100 comprises an inflow end 106, an outflow end 104, and a central longitudinal axis Ca extending in a direction from the inflow end 106 to the outflow end 104. In some instances, the inflow end 106 is the distal end of the prosthetic valve 100, and the outflow end 104 is the proximal end of the prosthetic valve 100. Alternatively, depending for example on the delivery approach of the valve, the inflow end can be the proximal end of the prosthetic valve, and the outflow end can be the distal end of the prosthetic valve.

The term “proximal”, as used herein, generally refers to a position, direction, or portion of a device or a component of a device, which is closer to the user (for example, during an implantation procedure) and further away from the implantation site.

The term “distal”, as used herein, generally refers to a position, direction, or portion of a device or a component of a device, which is further away from the user and closer to the implantation site.

The term “outflow”, as used herein, refers to a region of the prosthetic valve through which the blood flows through and out of the prosthetic valve 100.

The term “inflow”, as used herein, refers to a region of the prosthetic valve through which the blood flows into the prosthetic valve 100.

In the context of the present application, the terms “lower” and “upper” are used interchangeably with the terms “inflow” and “outflow”, respectively. Thus, for example, the lower end of the prosthetic valve is its inflow end and the upper end of the prosthetic valve is its outflow end.

The terms “longitudinal” and “axial”, as used herein, refer to an axis extending in the proximal and distal directions, unless otherwise expressly defined.

The prosthetic valve 100 comprises an annular frame 102 movable between a radially compressed state and a radially expanded state, and a valvular structure 140 mounted within the frame 102. The frame 102 can be made of various suitable materials, including plastically-deformable materials such as, but not limited to, stainless steel, a nickel based alloy (e.g., a cobalt-chromium or a nickel-cobalt-chromium alloy such as MP35N alloy), polymers, or combinations thereof. When constructed of a plastically-deformable materials, the frame 102 can be crimped to a radially compressed state on a balloon catheter (not shown), and then expanded inside a patient by an inflatable balloon or equivalent expansion mechanism. Alternatively or additionally, the frame 102 can be made of shape-memory materials such as, but not limited to, nickel titanium alloy (e.g., Nitinol).

In the example illustrated in FIGS. 1A-1B, the frame 102 is an annular, stent-like structure comprising a plurality of intersecting struts 108. In this application, the term “strut” 108 encompasses vertical struts, angled or curved struts, support posts, commissure windows, and any similar structures described by U.S. Pat. Nos. 7,993,394 and 9,393,110, which are incorporated herein by reference. A strut 108 may be any elongated member or portion of the frame 102. The frame 102 can include a plurality of strut rungs that can collectively define a plurality of cells 130 arranged in several cell rows. The frame 102 can have a cylindrical or substantially cylindrical shape having a constant diameter from the inflow end 106 to the outflow end 104 as shown, or the frame can vary in diameter along the height of the frame, as disclosed in U.S. Pat. No. 9,155,619, which is incorporated herein by reference.

Struts 108 comprise angled struts 110 and axial frame members 112. The term “axial frame member” refers to a strut or a component of the frame 102 that generally extends in an axial direction, while the term “angled strut” generally refers to a strut that can extend at an angle relative to an axial line intersecting therewith along a plane defined by the frame 102. It is to be understood that the term “angled strut” encompasses both linear angled struts and curved struts.

FIGS. 1A-1B show an exemplary prosthetic valve 100 that can be representative of, but is not limited to, a balloon expandable prosthetic valve. While the frame 102 illustrated in FIG. 1B is shown to include three rows of cells 130, it is to be understood that more or less rows of cells may be include in exemplary frame configurations.

Two or more struts 108 can intersect at junctions 134, which can be can be equally or unequally spaced apart from each other. The struts 108 may be pivotable or bendable relative to each other, so as to permit frame expansion or compression. For example, the frame 102 can be formed from a single piece of material, such as a metal tube, via various processes such as, but not limited to, laser cutting, electroforming, and/or physical vapor deposition, while retaining the ability to collapse/expand radially in the absence of hinges and like.

The frame 102 can further comprise a plurality of outflow apices 136 at the outflow end 104 of the frame, and a plurality of inflow apices 138 at the inflow end 106 of the frame. A plurality of intermediate junctions 134 are disposed between the inflow end 106 and outflow end 104.

In some examples, cells 130 are coupled to adjacent cells 130 within the same cell row, such as within a uppermost or proximal-most row of cells, via axial frame members 112. Axial frame members 112 can include, in some examples, axially extending commissure supports 116 and non-commissural axial struts 114. An axially extending commissure support 116 can include, in some examples, a window opening 118 between two axially extending sidewalls 120, wherein the commissure support 116 is configured to support a corresponding commissure 142 of the valvular structure 140. The axial frame members 112, including non-commissural axial struts 114 and axially extending commissure supports 116, can be parallel to each other and/or to the central longitudinal axis Ca of the prosthetic valve 100.

The terms “axially-extending commissure support” and “commissure support”, as used herein, are interchangeable, and refer to an axial frame member configured to support a commissure 142 attachable or attached thereto, and can include a window opening 118 defined therein. The terms “non-commissural axial strut” and “axial strut”, as used herein, are interchangeable, and refer to a an axial frame member configured to remain unattached to the valvular structure 140. That is to say, an axial struts 114 is not configured to mount a commissure, and may be devoid of a window opening.

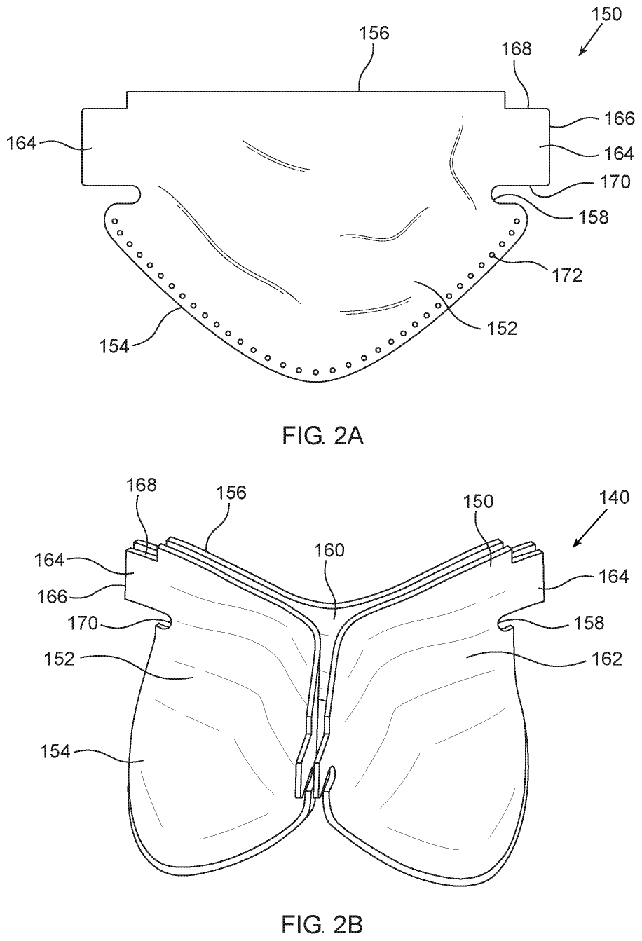

The valvular structure 140 comprises a plurality of leaflets 150 (e.g., three leaflets), positioned at least partially within the frame 102, and configured to regulate flow of blood through the prosthetic valve 100 from the inflow end 106 to the outflow end 104. FIG. 2A shows a front view in elevation of an exemplary leaflet 150, and FIG. 2B shows an exemplary valvular structure 140 comprised of a plurality of leaflets 150. While three leaflets 150 arranged to collapse in a tricuspid arrangement, are shown in the examples illustrated in FIGS. 1A and 2B, it will be clear that a prosthetic valve 100 can include any other number of leaflets 150. Adjacent leaflets 150 can be arranged together to form commissures 142 that are coupled (directly or indirectly) to respective portions of the frame 102, thereby securing at least a portion of the valvular structure 140 to the frame 102.

The leaflets 150 can be made from, in whole or part, biological material (e.g., pericardium), bio-compatible synthetic materials, or other such materials. Further details regarding transcatheter prosthetic valves, including the manner in which the valvular structures 140 can be coupled to the frame 102 of the prosthetic valve 100, can be found, for example, in U.S. Pat. Nos. 6,730,118, 7,393,360, 7,510,575, 7,993,394, 8,652,202, and 11,135,056, all of which are incorporated herein by reference in their entireties.

As shown for example in FIG. 2B, three separate leaflets 150 can collectively define the valvular structure 140 in some cases. As shown in FIG. 2A, a leaflet 150 has a movable leaflet body 152 defined between a cusp end portion 154 and an opposite free edge 156. The cusp end portion 154 can form, in some examples, a single scallop, such as by having a rounded edge concavely curved toward the inflow end 106 of the valve. In some examples, the leaflet 150 can further include a pair of generally oppositely-directed tabs 164 separating the cusp end portion 154 and the free edge 156. Each tab 164 can extend laterally away from the leaflet body 152 and terminate at a tab outer edge 166. Each tab 164 further defines a tab outflow edge 168 which is closer to the outflow end 104 of the frame, and a tab inflow edge 170 which is closer to the inflow end 106 of the frame. Each leaflet 150 further defines an inner surface 160, defined as the surface facing the central axis Ca of the prosthetic valve 100, and an outer surface 162, opposite thereto so as to face the frame 102.

When attached to a radially expandable frame, such as the frame 102 described above, the tabs 164 can extend through window openings 118 of commissure supports 116. Each window opening 118 can accommodate a pair of tabs 164 of two adjacent leaflets 150 (sometimes referred to as two tabs or a pair of tabs), such that the adjacent leaflets 150 form a commissure 142 between them. Each commissure 142 can be secured to the sidewalls 120 on both sides of the window opening 118.

The cusp end portion 154 of each leaflet 150 can be secured to the frame via one or more fasteners (for example, sutures). In some examples, the cusp end portion 154 of each leaflet 150 can be secured directly to the struts of the frame 102. For example, the cusp end portions 154 of the leaflets 150 can be sutured to selected angled struts 110 that generally follow the contour of the cusp edge portions of the leaflets, together defining a scalloped line of attachment. In some examples, as illustrated in FIG. 2A, the leaflet 150 can include a series of cusp pre-formed apertures 172 along the cusp end portion 154, through which a suture (or other fastener) can be passed to secure the leaflet 150 to the frame 102 along the scalloped line. In some examples, the leaflet 150 can be devoid of pre-formed apertures 172 along the cusp end portion 154.

The prosthetic valve 100 can further comprise at least one skirt or sealing member. In some examples, a prosthetic valve can include an inner skirt (not shown) secured to an inner surface of the frame 102, and configured to function, for example, as a sealing member to prevent or decrease perivalvular leakage. An inner skirt can further function as an anchoring region for the leaflets 150 to the frame 102, and/or function to protect the leaflets 150 against damage which may be caused by contact with the frame 102, for example during valve crimping or during working cycles of the prosthetic valve 100. In some examples, a prosthetic valve 100 can include an outer skirt 132 mounted on an outer surface of the frame 102, and configure to function, for example, as a sealing member retained between the frame 102 and the surrounding tissue of the native annulus against which the prosthetic valve is mounted, thereby reducing risk of paravalvular leakage (PVL) past the prosthetic valve 100.

Any of an inner skirt and/or outer skirt 132 can be made of various suitable biocompatible materials, such as, but not limited to, various synthetic materials (e.g., PET) or natural tissue (e.g. pericardial tissue). In some examples, an inner skirt comprises a single sheet of material that extends continuously around the inner surface of the frame 102. In some examples, the outer skirt 132 comprises a single sheet of material that extends continuously around the outer surface of the frame 102.

In some examples, the tab outflow edges 168 can be distally offset from the free edge 156 of the leaflet 150, as illustrated in FIG. 2A, forming an upper step-like configuration at the transition between the free edge 156 and each tab 164. In some examples, the tab outflow edges 168 can be axially aligned and continuous with the free edge 156 of the leaflet 150. In some examples, the leaflet 150 can further include indented sub-commissure portion 158.

FIG. 3 shows a perspective view of an exemplary delivery assembly 10 that includes a delivery apparatus 12 adapted to deliver a prosthetic device, which can be a prosthetic valve 100 described herein. The delivery apparatus 12 can include a handle 14 and at least one catheter extending therefrom, configured to carry a prosthetic valve 100 in a crimped state through the patient's vasculature. An exemplary delivery assembly 10 comprises an exemplary delivery apparatus 12 configured to carry a balloon expandable prosthetic valve. The delivery apparatus 12 can comprise a balloon catheter 20 having an inflatable balloon 22 mounted on its distal end. A prosthetic device, such as prosthetic valve 100, can be carried in a crimped state over the balloon catheter 20.

In some examples, a delivery apparatus 12 further comprises an outer shaft 18. Optionally, an outer shaft 18 of a delivery apparatus 12 can concentrically extend over the balloon catheter 20.

The outer shaft 18 and the balloon catheter 20 can be configured to be axially movable relative to each other. For example, a proximally oriented movement of the outer shaft 18 relative to the balloon catheter 20, or a distally oriented movement of the balloon catheter 20 relative to the outer shaft 18, can expose the prosthetic valve 30 from the outer shaft 18.

A delivery apparatus 12 can further include a nosecone 24 to facilitate advancement of the delivery apparatus 12 through the patient's vasculature to the site of treatment. A nosecone shaft (concealed from view in FIG. 3) can extend proximally from the nosecone 24 through a lumen of the balloon catheter 20, towards the handle 14.

In FIG. 3, a prosthetic valve 100 is mounted on the balloon 22 and is shown in a crimped state, providing prosthetic valve 100 with a reduced diameter for delivery to the heart via the patient's vasculature. While the prosthetic valve 100 is shown in FIG. 3 as being crimped or mounted on the balloon 22 for delivery to the treatment location, it should be understood that the prosthetic valve can be crimped or mounted at a location different from the location of balloon 22 (e.g., proximal to the balloon 22) and repositioned over the balloon at some time before inflating the balloon and deploying the prosthetic valve. This off-balloon delivery allows the prosthetic valve to be crimped to a lower profile than would be possible if the prosthetic valve was crimped on top of the balloon 22. The lower profile permits the clinician to more easily navigate the delivery apparatus (including the crimped prosthetic valve) through a patient's vasculature to the treatment location. The lower profile of the crimped prosthetic valve can be particularly helpful when navigating through portions of the patient's vasculature which are particularly narrow, such as the iliac artery.

The proximal ends of the balloon catheter 20, the outer shaft 18, and/or the nosecone shaft, can be coupled to the handle 14. During delivery, the handle 14 can be maneuvered by an operator (e.g., a clinician or a surgeon) to axially advance or retract components of the delivery apparatus 12, such as the nosecone shaft, the outer shaft 18, and/or the balloon catheter 20, through the patient's vasculature and/or along the target site of implantation, as well as to inflate the balloon 22 mounted on the balloon catheter 20, for example to expand a prosthetic valve 100 mounted on the balloon 22, and to deflate the balloon 22 and retract the delivery apparatus 12, for example once the prosthetic valve 100 is mounted in the implantation site.

The handle 14 can include a steering mechanism configured to adjust the curvature of a distal end portion of the delivery apparatus 12. In the illustrated example, the handle 14 includes an adjustment member, such as the illustrated rotatable knob 16a, which in turn is operatively coupled to the proximal end portion of a pull wire (not shown). The pull wire can extend distally from the handle 14 through the outer shaft 18 and has a distal end portion affixed to the outer shaft 18 at or near the distal end of the outer shaft 18. Rotating the knob 16a can increase or decrease the tension in the pull wire, thereby adjusting the curvature of the distal end portion of the delivery apparatus 12. Further details on steering or flex mechanisms for the delivery apparatus can be found in U.S. Pat. No. 9,339,384, which is incorporated by reference herein.

In some examples, the handle 14 can include an adjustment members such as the illustrated rotatable knob 16b, configured to adjust the axial position of the balloon catheter 20 relative to the outer shaft 18, for example for fine positioning at the implantation site. The handle can include additional knobs to control additional components of the delivery apparatus 12. Further details on the delivery apparatus 12 can be found in PCT Application No. PCT/US2021/047056, which is incorporated by reference herein.

A prosthetic valve 100 can be carried by the delivery apparatus 12 during delivery in a crimped state, and expanded, for example by balloon inflation, to secure it in a native heart valve annulus (such as an aortic annulus) or against a previously implanted prosthetic valve (for example, during valve-in-valve implantation procedures). In some examples, the balloon 22 is secured to a distal end portion of the balloon catheter 20 at its proximal end, while the balloon's distal end can be coupled, directly or indirectly, to another component of the delivery apparatus 12, such as the nosecone 24 or nosecone shaft.

Balloon 22 is configured to transition between a deflated and inflated states. Upon reaching the site of implantation, the balloon 22 can be inflated to radially expand the prosthetic valve 100. Once the prosthetic valve 30 is expanded to its functional diameter within a native annulus, the balloon 22 can be deflated, and the delivery apparatus 12 can be retrieved from the patient's body.

In some examples, the delivery apparatus 12 with the prosthetic valve 30 assembled thereon, can be packaged in a sterile package that can be supplied to end users for storage and eventual use. In some examples, when the leaflets of the prosthetic valve are made from, or include at least an inner core made from, bovine pericardium tissue or other natural or synthetic tissues, the leaflets can be treated during the manufacturing process so that they are completely or substantially dehydrated and can be stored in a partially or fully crimped state without a hydrating fluid. In this manner, the package containing the prosthetic valve and the delivery apparatus, can be free of any liquid. Methods for treating tissue leaflets for dry storage are disclosed in U.S. Pat. Nos. 8,007,992 and 8,357,387, both of which documents are incorporated herein by reference.

Commissures 142 of a valvular structure 140 may be subject to various stresses both during the delivery of a prosthetic valve and after the prosthetic valve is deployed in a patient's vasculature. For example, the frame may apply stresses to the leaflet during radial expansion of the frame, and the opening and closing of the valvular structure under the flow of blood may further stress the leaflets. Particularly, when load-bearing sutures are passed through the leaflets, they provide stress concentrators in the leaflet material. To mitigate stresses on the leaflets at the commissures, known prosthetic valves can include multiple layers of leaflet material and reinforcing fabric forming each commissure. While durable, the various layers of material forming the commissures can limit the crimp profile of the prosthetic valve.

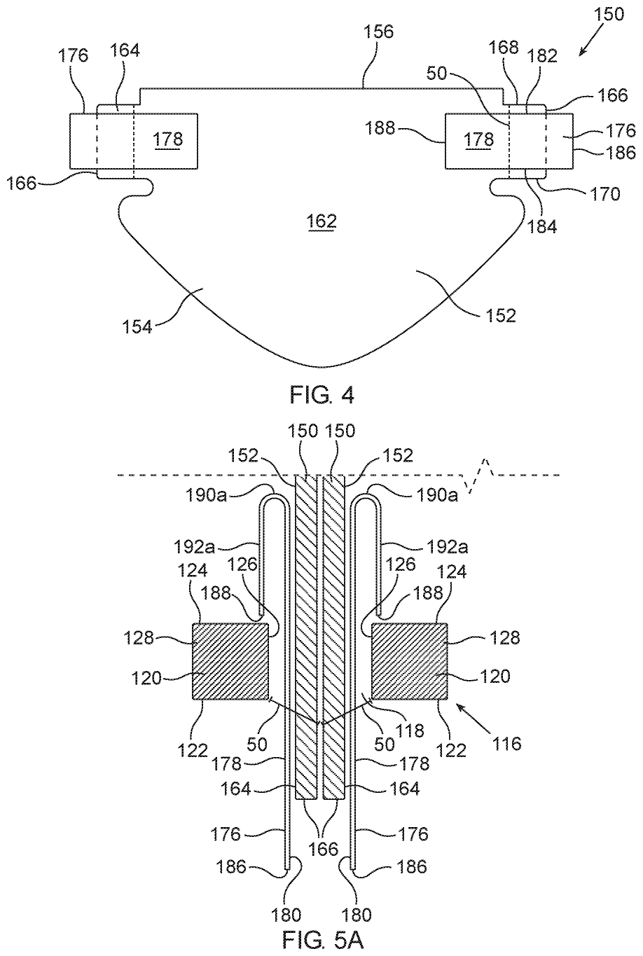

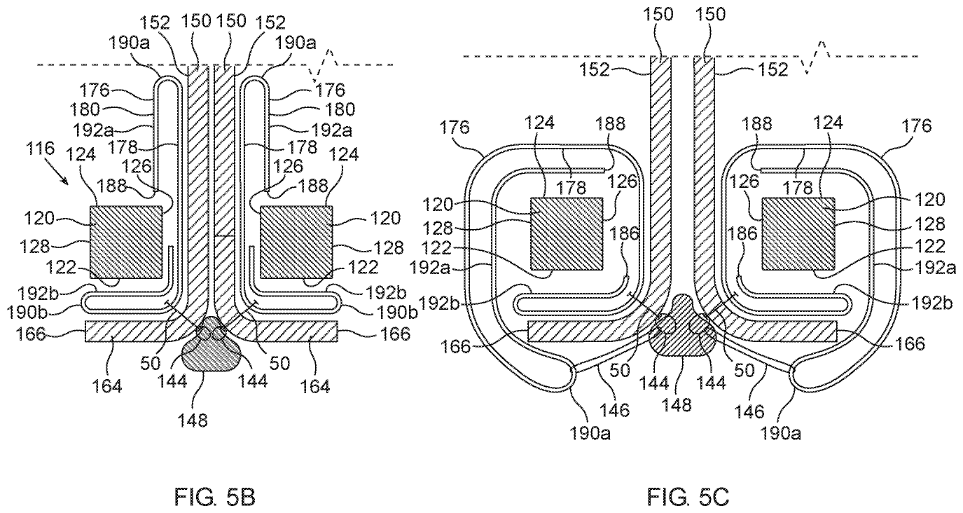

FIGS. 4-5C show an exemplary commissure 142 that includes reinforcement members, termed herein reinforcement posts 176, stitched to respective tabs 164 along two or more suture lines, wherein each commissure tab can be secured to a frame portion with the main load bearing sutures in the form of stitches that extend through respective stitches of two suture lines, rather than through the leaflet itself. Advantageously, this removes stress concentrators from the leaflet material, thereby permitting the use of fewer layers of material to form the commissures. This, in turn, decreases the crimp profile (that is, the diameter of the prosthetic heart valve in the radially compressed configuration) of the prosthetic heart valve. In this way, a commissure assembly with a narrow, yet sturdy configuration is provided.

FIG. 4 illustrates a front elevation view of a leaflet 150 that includes reinforcement posts 176 attached to the tabs 164. In some examples, the reinforcement post 176 can be made of any of various biocompatible fabrics, such as polyethylene terephthalate (PET) fabric, or non-fabric materials, such as polyurethane, silicone, or other polymers. In some examples, the reinforcement posts 176 can be made of natural tissue, such as pericardium. In some examples, the reinforcement posts 176 can comprises a combination of natural tissue and a synthetic material.

Reinforcement post 176 can define a post outflow edge 182 which is closer to the outflow end 104 of the frame 102 and/or to the tab outflow edge 168, and a post inflow edge 184 which is closer to the inflow end 106 of the frame 102 and/or to the tab inflow edge 170. When reinforcement post 176 is laid flat on a surface of the leaflet, prior to being optionally folded over itself, the reinforcement post 176 extends from a post inner edge 188, which is closer to the leaflet body 152, to a post outer edge 186 which can be closer and/or positioned past the tab outer edge 166, in the flattened configuration of the reinforcement posts 176 shown in FIG. 4 to be laid flat over the leaflet 150, prior to forming any overfolded portions of the reinforcement post. Stated otherwise, the post inner edge 188 is positioned radially inwards to the post outer edge 186 when the reinforcement post 176 is laid over a surface of the leaflet 150.

As shown in FIG. 4, in some examples, the reinforcement post 176 can cover a portion of the respective tab 164. In some examples, the reinforcement post 176 can cover the entirely of the respective tab 164. In some examples, the reinforcement post 176 can be centered relative to the respective tab 164 in an axial direction, such that the post outflow edge 182 and the post inflow edge 184 are equally distanced from the respective tab outflow edge 168 and tab inflow edge 170. In some examples, the reinforcement post 176 can be offset in an axial direction relative to the respective tab 164.

The reinforcement member 176 can define a first side 178 and a second side 180. As shown in FIG. 4, when the reinforcement member 176 is laid in a flat unfolded (or pre-folded) configuration thereof over the outer surface 162 of the leaflet 150, the first side 178 is defined as the surface facing away from the leaflet 150 and its outer surface 162, while the second side 180 (hidden in FIG. 4) defines the surface facing and at least partially contacting the outer surface 162 of the leaflet 150.

The reinforcement post 176 can be attached to the leaflet 150, such as to the tab 164 of the leaflet 150, by one or more suture lines (also referred to as vertical stitch lines 50). In the example illustrated in FIG. 4, a vertical stitch line 50 can extend through the reinforcement post 176 and the tab 164, thereby securing the reinforcement post 176 to the respective tabs 164. In some examples, a vertical stitch line 50 can form a plurality of in-and-out stitches that extend out of the first side 178 of the respective reinforcement post 176, as well as out of an opposite inner surface 160 of the respective tab 164. While not shown in FIG. 4, it is to be understood that in some configuration, additional vertical stitch lines 50 can extend through layers of overfolded portions of the reinforcement post 176, without necessarily extending through the leaflet 150.

In some examples, the locations of the vertical stitch lines 50 on the reinforcement post 176 can be selected based geometries and/or sizes of the window opening 118, the sidewalls 120, the tabs 164, and/or a desired commissure configuration. In some examples, a length of the reinforcement post 176 and/or a position of the reinforcement post 176 relative to the tabs 164 can be selected based geometries and/or sizes of the window opening 118, the sidewalls 120, the tabs 164, and/or a desired commissure configuration. In some examples, a reinforcement post 176 can extend past the tab 164, such that the post outer edge 186 is positioned radially away from the tab outer edge 166, as illustrated in FIG. 4.

The arrangement of the tabs 164, the reinforcement posts 176, and the vertical stitch line 50, can allow the leaflets 150 to be attached to a commissure support 116 with minimal need for additional leaflet material to reinforce attachment points, while avoiding load-bearing stitches that extend through the leaflet or at least minimizing load-bearing on stitches that extend through the leaflet, as described in greater detail herein.

An exemplary first stage of attachment of paired tabs 164 of adjacent leaflets 150 to sidewalls 120 of a commissure support 116 is illustrated in FIG. 5A. An exemplary second stage of attachment of the paired tabs 164 of adjacent leaflets 150 to sidewalls 120 of the commissure support 116 is illustrated in FIG. 5B. An exemplary third stage of attachment of the paired tabs 164 of adjacent leaflets 150 to sidewalls 120 of the commissure support 116 is illustrated in FIG. 5C, and shows an exemplary configuration for a commissure 142 that can be formed with the leaflets 150 including the reinforcement posts 176. It will be appreciated that FIG. 5A-5C are not drawn to scale to improve clarity in the depiction of the interaction between the components and/or the arrangement of the components, such as the leaflet 150, the reinforcement post 176, the vertical stitch line 50, the sidewalls 120 of the commissure support 116, and the connecting sutures (discussed below).

Each of the sidewalls 120 on both sides of the window opening 118 of a commissure support 116 defines a sidewall inner surface 124 oriented radially inwards, towards the longitudinal central axis Ca, an opposite sidewall outer surface 122, oriented away from the longitudinal central axis Ca, a lateral internal surface 126, facing the window opening 118, and an opposite lateral external surface 128, oriented away from the window opening 118.

To attach the tabs 164 of the leaflets 150 and the reinforcement posts 176 to a sidewall 120 of the commissure support 116, the tabs 164 and the reinforcement posts 176 can be inserted through the window opening 118 from inside the frame 102 and can extend radially outward past the sidewalls 120 such that a portion of each tab 164 and reinforcement post 176 is outside the frame 102. As shown in FIG. 5A, at the first stage of formation, the vertical stitch line 50 can be positioned radially outwards of the sidewalls 120 or the commissure support 116 (outside of the frame 102). The reinforcement post 176 can be positioned against a lateral internal surface 126 of each of the sidewalls 120. Inner edges 188 of the reinforcement posts 176 can be folded outward (away from the leaflet body 152) to create folded portions 192a along fold lines 190a in the reinforcement posts 176 in portions thereof that are interior of the frame 102.

In the second stage of attachment shown in FIG. 5B, a wedge member 148 can be positioned between the adjacent tabs 164 at a location where they exit the window opening 118, and first securing loops 144 can attach the wedge member 148 to each of the vertical stitch lines 50. When the wedge member 148 is positioned between the adjacent tabs 164, the tabs 164 are compressed between the sidewalls 120 and the wedge member 148, preventing slippage of the tabs 164 relative to the sidewalls. The wedge member 148 can comprise, for example, a piece of fabric or a relatively thick suture (for example, a braided suture, such as an Ethibond suture).

In some examples, the vertical stitch lines 50 can be positioned such that they are aligned with corners of the sidewalls 120 between the lateral internal surface 126 and the sidewall outer surface 122 of the respective sidewall, and are adjacent to the wedge member 148. In some examples, first securing loops 144 (that is, wedge securing loops 144) can each extend from one of the vertical stitch lines 50 and through the wedge member 148. When the first securing loops 144 are tightened, the wedge member 148 can be pressed radially inwards against the tabs 164, compressively securing the tabs 164 between the wedge member 148 and the surfaces 122, 126 of the sidewalls 120. In some examples, a single securing loop can extend to both of the vertical stitch lines 50 and through the wedge member 148.

The securing loops 144 can each pass through at least one stitch in one of the vertical stitch lines 50. In some examples, the securing loops 144 can each extend only through a single stitch of each of the vertical stitch lines 50. In some examples, the securing loops 144 can each extend through multiple stitches of the vertical stitch lines 50, such that the securing loop 144 forms multiple loops extending continuously back and forth between the vertical stitch line 50 and the wedge member 148 along the length of the respective vertical stitch lines 50 in a helical or zig-zag pattern. In some examples, a plurality of discrete securing loops 144 can be formed, each of which extends through a respective stitch and the wedge member 148 along the length of the vertical stitch lines 50.

In some examples, outer edges 186 (opposing the inner edges 188) of the reinforcement posts 176 can be folded outwardly (away from the leaflet body 152) to form second folded portions 192b along second fold lines 190b, exterior of the frame 102. In some examples, the outer edges 186 can remain unfolded, and the folded portions 192b can be excluded from the commissure configuration.

After insertion of the tabs 164 and the reinforcement posts 176 through the window opening 118 (and formation of the folded portions 192b, if included), the tabs 164 and the reinforcement posts 176 can be wrapped over the sidewalls 120 to extend over the sidewall outer surfaces 122. In some examples, the folded portions 192b of the reinforcement posts 176 can be positioned on the sidewall outer surfaces 122 and can be disposed between the tabs 164 and the outer surface 122 of the respective sidewall 120. In some examples, the bends in the tabs 164 can be right angle bends or approximately right-angle bends (that is, the bends can each be 90-degree bends or approximately 90-degree bends).

As shown in FIG. 5C, portions of the reinforcement posts 176 on the interior of the frame 102 (such as, portions of the reinforcement posts 176 on the interior of the frame 102 in FIGS. 5A and 5B) can be wrapped over the sidewalls 120 so that they extend around inner surface 128 and lateral external surface 128 and over at least a portion of the outer surface 122 of the respective sidewall 120. In some examples, the post inner edges 188 can be aligned with corners at intersections between the lateral internal surfaces 126 and inner surfaces 124 of the sidewalls 120, such that the folded portions 192a wrap around the sidewalls 120 and fold lines 190a of the folded portions 192a are exterior of the frame 102. In some examples, at least a portion of the exterior surfaces of the tabs 164 (exterior of the frame 102) can be covered by the reinforcement posts 176. In some examples, portions of the tabs 164 that extend over the sidewall outer surfaces 122 can be sandwiched between the first and second folded portions 192a, 192b (that is, between four layers of the reinforcement posts 176).

In some examples, securing loops 146 (also referred to as “an outer securing loops”) can extend between the fold lines 190a of the folded portions 192a and the securing loops 144. In some examples, a single securing loop 146 can extend between the fold lines 190a of the folded portions (rather two loops).

The securing loops 146 can pass through at least one stitch of each of the securing loops 144. In some examples, securing loops 146 can pass through an upper stitch and a lower stitch of the securing loops 144 that correspond to a height of the reinforcement post. In some examples, the securing loops 146 can additionally or alternatively extend through the wedge member 148.

When the securing loops 146 are tightened, the reinforcement posts 176 can be fixed at the exterior side of the commissure support 116 and resist and/or prevent movement of the reinforcement posts 176 relative to commissure support 116. Additionally, the wedge member 148 can be further pressed radially inwards against the tabs 164, further compressively securing the tabs 164 between the wedge member 148 and the sidewall outer surfaces 122.

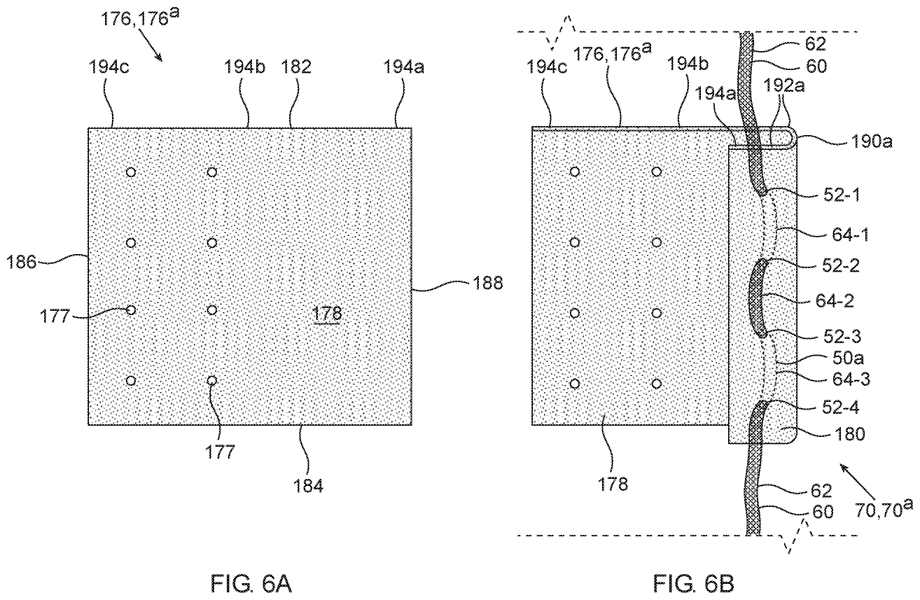

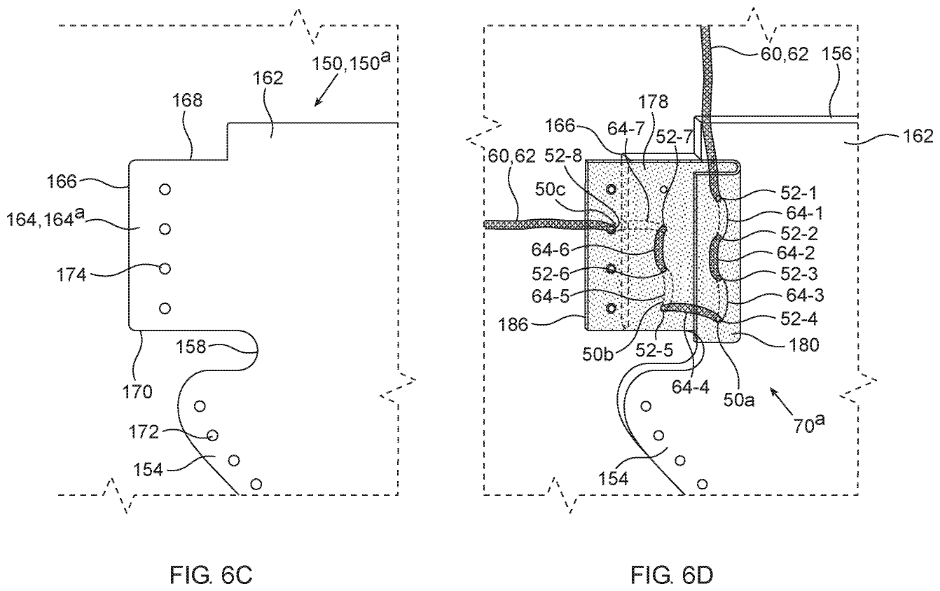

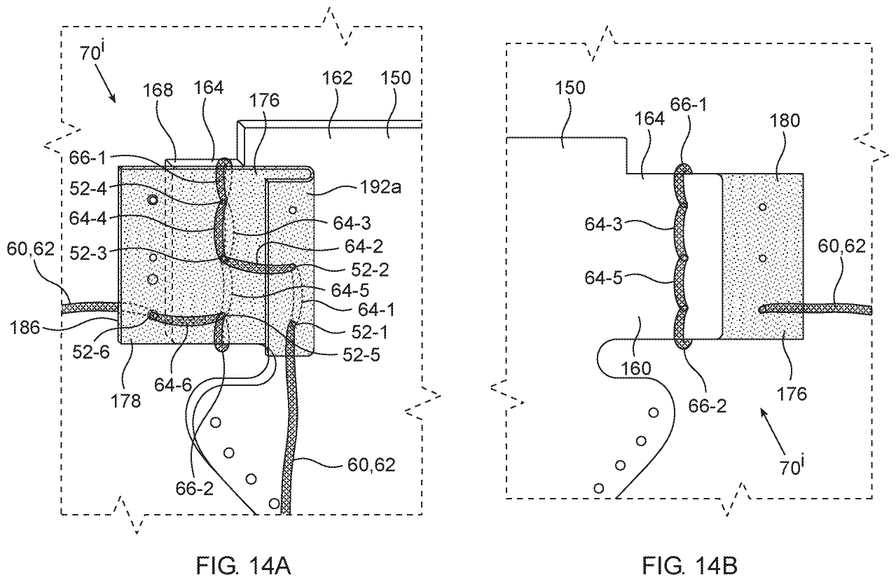

In some examples, a prosthetic valve 100 comprises multiple commissure supports 116 for multiple leaflets 150, forming a corresponding number of commissures assembled in the fashion described above in relation to FIGS. 5A-5C or according to any of the stitching patterns described below in relation to FIGS. 6A-17B. For example, a prosthetic heart valve can have two or more leaflets 150, such as three leaflets 150, or more than three leaflets 150, wherein each leaflet 150 comprises two tabs 164, and a matching number of commissure supports 116 having window openings 118 receive the tabs 164 to form a number of commissures equal to the number of leaflets, such as two commissures 142, three commissures 142, or more than three commissures 142.

In this way, one or more relatively narrow commissure assemblies that are nonetheless durable can be assembled. Because the tensioning forces securing the tabs 164 to the sidewalls 120 are borne at least by the first securing loop 144 and/or the second securing loop 146, and because the first securing loop 144 and the second securing loop 146 do not pass through the leaflet 150, the securing loops 144, 146 do not introduce any holes or apertures in the leaflet 150. Accordingly, no stress concentrators are added to the leaflet, thereby improving leaflet durability.

Also, because no suture line attaches any leaflet to itself or to another leaflet, the window opening 118 between both sidewalls 120 need only to accommodate tabs 164 of two adjacent leaflets, along with a relatively thin layer of the reinforcement post 176 attached to each tab 164, such that a single tab 164 is folded around the corresponding sidewall 120. Thus, any assembly comprising the window opening 118 of a commissure support 116, the tabs 164, and the reinforcement posts 176 as described herein, for example, may have a reduced profile compared to known prosthetic valves. This, in turn, reduces the overall crimp profile of the prosthetic valve. In some examples, such as when the leaflets 150 are made of tissue (e.g., pericardium), the window opening 118 can have narrower dimensions than a combined thickness of two relaxed leaflets 150 (or tabs 164 thereof) positioned side-by-side, taking advantage of tissue compressibility to allow the tissue material to be compressed as it is passed through the window opening 118.

While the examples described herein pertain to a commissure supports 116 having window openings 118 formed between two rectangular sidewalls 120, it is understood that the geometry of the sidewalls 120 can be varied according to the needs of the frame. For example, the sidewalls 120 can have a circular, ovular, or polygonal cross section. In such examples, the reinforcement posts 176 and the tabs 164 can be wrapped or folded around the circumference, perimeter, or any number of sides of the window sidewalls.

Additional exemplary leaflets including attached reinforcement posts, exemplary stitching patterns along the reinforcement posts and/or commissure attachment, and exemplary commissure configurations, are shown in FIGS. 6A-17B. It will be appreciated that, in some examples, the exemplary leaflets, reinforcement posts, stitching patterns, and commissure attachment configurations of FIGS. 6A-17B can have one or more features of the examples described above with respect to FIGS. 1-5C. In some examples, the exemplary leaflets, reinforcement posts, stitching patterns, and commissure attachment configurations of FIGS. 6A-17B can have one or more different features relative to the examples of FIGS. 1-5C, as described below.

FIG. 6A is a front view in elevation of an exemplary reinforcement post 176^{a}. Various exemplary implementations for leaflets, reinforcement posts, commissures and stitching patterns can be referred to, throughout the specification, with superscripts, for ease of explanation of features that refer to such exemplary implementations. It is to be understood, however, that any reference to structural or functional features of any leaflet, reinforcement post, commissure or stitching pattern, without a superscript, refer to these features being commonly shared by all specific exemplary implementations that can be also indicated by superscripts. In contrast, features emphasized with respect to an exemplary implementation of any leaflet, reinforcement post, commissure or stitching pattern, referred to with a superscript, may be optionally shared by some but not necessarily all other exemplary implementations.

For example, reinforcement post 176^{a} illustrated in FIG. 6A is an exemplary implementation of reinforcement post 176, and thus can include any of the features described for reinforcement post 176 throughout the current disclosure, except that the reinforcement post 176^{a} can further include post pre-formed apertures 177 through which a suture can extend during stages of commissure formation. In some examples, a plurality of post pre-formed apertures 177 can be arranged in one or more series of apertures radially aligned with each other. The term “radially aligned”, as used herein with respect to positions along a reinforcement post 176, refer to positions which are equally distanced from a side edge of the reinforcement post 176, such as the post outer edge 186 or the post inner edge 188. Each series of radially aligned apertures 177 can include a plurality of apertures 177 equally or non-equally spaced from each other along the height of the reinforcement post 176, between the post outflow edge 182 and the post inflow edge 184. Apertures 177 can be pre-formed in the reinforcement post 176 by one or more of punching (e.g., punching a hole into the leaflet and removing the punched-out tissue portions), laser heating, and/or by using a femtolaser to form the apertures.

In some examples, each series of radially aligned apertures 177 can define a vertical stitch line 50 when a suture is passed to form one or more stitches axially extending between the post outflow edge 182 and the post inflow edge 184. In the example illustrated in FIG. 6A, two series of radially aligned apertures 177 are shown, each including four post pre-formed apertures 177 which are axially spaced from each other. It is to be understood that a reinforcement post 176 can include any other number of series of radially-aligned apertures 177, such as a single series or more than two series of radially-aligned apertures 177, and that each series of radially aligned apertures 177 can include any other number of apertures 177 that are axially spaced from each other.

Post pre-formed apertures 177 can mark penetration points through which a suture can extend through one or more layers of the reinforcement post 176, and optionally through the thickness of a corresponding tab 164 to which the reinforcement post 176 can be secured. While a plurality of apertures 177 can be pre-formed in a reinforcement post 176, it is to be understood that a suture is not necessarily passed through all apertures 177. For example, different stitching patterns can include insertion of a suture through one, some, but not necessarily all of the apertures 177.

In some examples, pre-formed apertures 177 can be replaced with other forms of marking the positions through which a suture can be passed. For example, printed markers such as ink-dots and the like can be included to visibly mark penetration points through which a suture can be passed following a desired stitching pattern. In some examples, a reinforcement post 176 can be devoid of pre-formed apertures 177 or other markers indicating specific potential suture penetration points.

In some examples, a reinforcement post 176 can generally include several post sections 194 which are continuous regions of the reinforcement post 176 that can be overfolded or otherwise arranged during commissure formation. The post sections 194 can include, in some examples, a first post section 194a extending from the post inner edge 188, a second post section 194b continuously extending from the first post section 194a, and a third post section 194c continuously extending from the second post section 194b to the post outer edge 186.

FIGS. 6B and 6D show exemplary stages of forming a stitching pattern 70 along a reinforcement post 176 secured to a tab 164. A stitching pattern 70 refers to a path through which a suture 60 is passed through one or more layers of a reinforcement post 176 and/or a tab 164, and includes a plurality of subsequent stitches 64 and/or loops 66. A stitch 64 is defined as a portion of the suture 60 extending between two adjacent penetration points 52. A penetration point 52 is a point through which a suture 60 extends through one or more layers of the reinforcement post 176, and/or through the thickness of the tab 164. A stitch 64 can be disposed over a surface of the reinforcement post 176 or a surface of the tab 164. For example, a stitch 64 can be disposed over a first side 178 of the reinforcement post 176, over a second side 180 of the reinforcement post 176, over an inner surface 160 of the tab 164, or over an outer surface 162 of the tab 164.

A loop 66 is defined as a portion of the suture 60 extending full-circle either through two adjacent penetration points 52 or through a single penetration point 52 and over an edge of the reinforcement post 176 and/or the tab 164. A loop 66 extending between two adjacent penetration points 52 can be formed by passing the suture through a first penetration point 52, towards and through an adjacent second penetration point 52, and back towards and through the first penetration points 52, such that the formed loop 66 in such a case can be also regarded as a combination of two continuous stitches aligned with each other and disposed on opposite surfaces of the corresponding reinforcement post 176 and/or tab 164. A loop 66 extending through a single penetration point 52 can be formed by passing the suture through the penetration point 52, along one surface of the reinforcement post 176 and/or tab 164, over one or more edges, such as the post outflow edge 182 and/or the tab outflow edge 168, or such as the post inflow edge 184 and/or the tab inflow edge 170, and over the opposite surface of the tab 164 and/or reinforcement post 176 towards and through the same penetration point 52.

Stitches 64 can extend in various orientations. A vertical stitch 64 refers to a stitch extending between two radially-aligned penetration points which are axially spaced from each other, such as in a direction from the post outflow edge 182 towards the post inflow edge 184, or from the tab outflow edge 168 towards the tab inflow edge 170. A horizontal stitch 64 refers to a stitch extending between to axially-aligned penetration points which are radially (or laterally) spaced from each other, generally perpendicular to the direction of a vertical stitch. A diagonal stitch 64 refers to a stitch extending between two penetration points which are both radially and axially offset from each other.

In some examples, a tab 164 of a leaflet 150 can include one or more tab pre-formed apertures 174. FIG. 6C is a front view in elevation of a portion of an exemplary leaflet 150^{a}, which is similar to any example described herein for leaflet 150, except that the tab 164^{a} of leaflet 150^{a} further includes tab pre-formed apertures 174 through which a suture can extend during stages of commissure formation. In some examples, a plurality of tab pre-formed apertures 174 can be arranged in one or more series of apertures radially aligned with each other. The term “radially aligned”, as used herein with respect to positions along a tab 164, refer to positions which are equally distanced from the tab outer edge 166. Each series of radially aligned apertures 174 can include a plurality of apertures 174 equally or non-equally spaced from each other along the height of the tab, between the tab outflow edge 168 and the tab inflow edge 170. Apertures 174 can be pre-formed in the reinforcement post 176 by one or more of punching (e.g., punching a hole into the leaflet and removing the punched-out tissue portions), laser heating, and/or by using a femtolaser to form the apertures

In some examples, each series of radially aligned apertures 174 can be defined along a vertical stitch line 50 when a suture is passed to form one or more stitches axially extending between the tab outflow edge 168 and the tab inflow edge 170. In the example illustrated in FIG. 6C, a single series of radially aligned apertures 174 are shown, including four tab pre-formed apertures 174 which are axially spaced from each other. It is to be understood that a tab 164 can include any other number of series of radially-aligned apertures 174, such as two or more series of radially-aligned apertures 174, and that each series of radially aligned apertures 174 can include any other number of apertures 174 that are axially spaced from each other.

Tab pre-formed apertures 174 can mark penetration points through which a suture can extend through the thickness of the tab 164, and optionally through one or more layers of a reinforcement post 176 secured thereto. In some examples, at least some of the tab pre-formed apertures 174 are aligned with at least some of the post pre-formed apertures 177. For example, a reinforcement post 176 can be laid over the corresponding tab 164 with one or more series of post pre-formed apertures 177 aligned with one or more series of tab pre-formed apertures 174. In some examples, post pre-formed apertures 177 and/or of tab pre-formed apertures 174 can assist in proper positioning of the reinforcement post 176 along the respective tab 164. A suture passing through a series of radially-aligned apertures 174 and 177 can define a vertical stitch line 50 securing the reinforcement post 176 to the tab 164, for example.

While a plurality of apertures 174 can be pre-formed in a tab 164, it is to be understood that a suture is not necessarily passed through all apertures 174. For example, different stitching patterns can include insertion of a suture through one, some, but not necessarily all of the apertures 174.

In some examples, pre-formed apertures 174 can be replaced with other forms of marking the positions through which a suture can be passed. For example, printed markers such as ink-dots and the like can be included to visibly mark penetration points through which a suture can be passed following a desired stitching pattern. In some examples, a reinforcement post 176 can be devoid of pre-formed apertures 177 or other markers indicating specific potential suture penetration points.

FIGS. 6B and 6D show stages in the formation of an exemplary stitching pattern 70^{a}. In some examples, as shown in FIG. 6B, the inner edge 188 of the reinforcement post is folded outwards (away from the leaflet 150 and its tab 164 when the reinforcement post 176 is secured to a tab 164) to create a folded portion 192 a along a fold line 190a, which can be also referred to as a first folded portion 192a and a first fold line 190a. The first post section 194a can serve as the section of the reinforcement post 176 forming the folded portion 192a. When the folded portion 192 is created, the position at which the post inner edge 188 is folded over the first side 178 of the reinforcement post 176 can generally designate the position from which the second post section 194b extends towards the post outer edge 186.

A first vertical stitch line 50a can include a plurality of in-and-out stitches 64 extending through the two folded layers along the folded portion 192a of the reinforcement post 176, to maintain the folded portion 192 in an overfolded configuration. As further shown in FIG. 6B, the in-and-out stitches 64 of the first vertical stitch line 50a extend through both layers of the folded portion 192a of the reinforcement post 176, without extending through a leaflet 150.

A first vertical stitch line 50a of a stitching pattern 70^{a}, illustrated in FIG. 6B, includes a first vertical stitch 64-1 extending between a first penetration point 52-1 and a second penetration point 52-2, a second vertical stitch 64-2 extending between the second penetration point 52-2 and a third penetration point 52-3, and a third vertical stitch 64-3 extending between the third penetration point 52-3 and a fourth penetration point 52-4, such that the first and third vertical stitches 64-1, 64-3, are disposed over a surface of the folded portion 192a facing the leaflet 150, and the second vertical stitch 64-2 is disposed over a surface of the folded portion 192a facing away from the leaflet 150.

The first penetration point 52-1 is distally offset from the post outflow edge 182, the second penetration point 52-2 is distal to the first penetration point 52-1, the third penetration point 52-3 is distal to the second penetration point 52-2, and the fourth penetration point 52-4 is distal to the third penetration point 52-3 and is proximally offset from the post inflow edge 184. As further shown in FIG. 6B, tails 62 of the suture 60 can extend from the first penetration point 52-1 and fourth penetration point 52-4 along an outer surface of the folded portion 192a.

As shown in FIG. 6D for example, the reinforcement post 176 can be secured to the tab 164 of a leaflet 150 via subsequent in-and-out stitches 64 that extend through the reinforcement post 176 and the tab 164. The reinforcement post 176 can be laid over the leaflet 150 and its tab 164 such that at least part of the second side 180 of reinforcement post 176 contacts at least part of the outer surface 162 of the tab 164. In some examples, the first side 178 of the reinforcement post 176 along the second post section 194b contacts and/or covers at least part of the outer surface 162 of the tab 164. The folded portion 192a can extend next to the leaflet body 152 and/or the tab 164. In some examples, a second vertical stitch line 50b extends along the second post section 194b and includes one or more in-and-out stitches 64 configured to secure the reinforcement post 176 to the tab 164.

In some examples, the third post section 194c is a section of the reinforcement post 176 extending past the outer edge 166 of the tab 164. Thus, a third post section 194c can extend between the position of the tab outer edge 166 and the post outer edge 186. In some examples, a third vertical stitch line 50c can be formed along the third post section 194c.

FIG. 6D shows a continuation of the stitching pattern 70^{a} of FIG. 6B. As shown, the stitching pattern 70^{a} includes a fourth stitch 64-4, which is a horizontal stitch, extending between the fourth penetration point 52-4 and a fifth penetration point 52-5. The fourth penetration point 52-4 can be part of the first vertical stitch line 50a, and the fifth penetration point 52-5 can be part of the second vertical stitch line 50b, such that the horizontal stitch 64-4 can bridge between the first 50a and second 50b vertical stitch lines. The horizontal stitch 64-4 is disposed over outer surfaces of the reinforcement post 176, extending over part of the folded portion 192a, the post inner edge 188, and part of the second post section 194b.

It is to be understood that the terms “first”, “second”, “third”, and so on, with respect to any of the stitches 64, loops 66, and/or penetration points 52 described with respect to any stitching pattern 70 disclosed throughout the specification and the claims, are not intended to designate a specific order to stitching, and that such stitches or loops can be formed in any desired order to achieve a final stitching pattern configuration.

As mentioned above, the stitches 64 along the first vertical stitch line 50a extend only through layers of the folded portion 192 without extending through the leaflet material. Thus, when the reinforcement post 176 is secured to the tab 164, one or more stitches 64 of the first vertical stitch line 50a, such as stitches 64-1 and 64-3 in the example of stitching pattern 70^{a} shown in FIG. 6D, are disposed between an inner surface of the reinforcement post 176 and the outer surface 162 of the leaflet 150.

In some examples, overfolding the first post section 194a to form the folded portion 192 and threading a suture 60 therethrough to form stitches 64 along a first vertical stitch line 50a can be performed prior to securement of the reinforcement post 176 to the tab 164, as illustrated, for example, in FIG. 6B, after which the reinforcement post 176, including folded portion 192a, is laid over the leaflet 150 and one of the tails 62 of the suture 60 extending from the first vertical stitch line 50a can be used to form subsequent stitches 64 to secure the reinforcement post 176 to the tab 164.

It is to be understood that any reference to an outer side or surface of the reinforcement post 176 refers to surfaces of the reinforcement post 176 facing away from the leaflet 150, while an inner side or surface of the reinforcement post 176 refers to a surface of the reinforcement post 176 facing the leaflet 150. Thus, an inner surface of the reinforcement post 176 can include at least part of the second side 180 of the reinforcement post 176. When a reinforcement post 176 is laid flat over leaflet 150, prior to forming any folded portion 192 thereof, the outer surface of the reinforcement post 176 can include solely the first side 178 of the reinforcement post 176.

When the reinforcement post 176 includes a first folded portion 192a, the overfolded part of the reinforcement post 176 extending between the fold line 190a and the post inner edge 188 is inverted, such that the second side 180 of this inverted part constitutes part of the outer surface of the reinforcement post 176. Thus, in such a case, the outer surface of the reinforcement post 176 can be comprises of part of the first side 178 of the reinforcement post 176, such as along at least part of the second post section 194b, and part of the first side 178 along the overfolded part of the folded portion 192a extending between the fold line 190a and the post inner edge 188.

As further shown in FIG. 6D, the stitching pattern 70^{a} further includes two in-and-out stitches along a second vertical stitch line 50b, namely a fifth vertical stitch 64-5 extending between the fifth penetration point 52-5 and a sixth penetration point 52-6, and a sixth vertical stitch 64-6 extending between the sixth penetration point 52-6 and a seventh penetration point 52-7, and a second penetration point 52-2, such that the fifth vertical stitch 64-5 is disposed over the inner surface 160 of the tab 164, and the sixth vertical stitch 64-6 is disposed over the outer surface or first side of the reinforcement post 176.

The stitching pattern 70^{a} can further include a seventh stitch 64-7, which can be a horizontal stitch, extending between the seventh penetration point 52-7 and an eighth penetration point 52-8. The seventh penetration point 52-7 can be part of the second vertical stitch line 50b, and the eighth penetration point 52-8 can be part of a later formed third vertical stitch line 50c, such that the horizontal stitch 64-7 can bridge between the second 50b and third 50c vertical stitch lines. The horizontal stitch 64-7 is disposed over the inner surface 160 of the tab 164, the tab outer edge 162, and an inner surface or second side 180 of reinforcement post 176, such as along part of the third post section 194c. A tail 62 of the suture 60 can extend from the eighth penetration point 52-8, for example along the first side 178 of the reinforcement post 176.

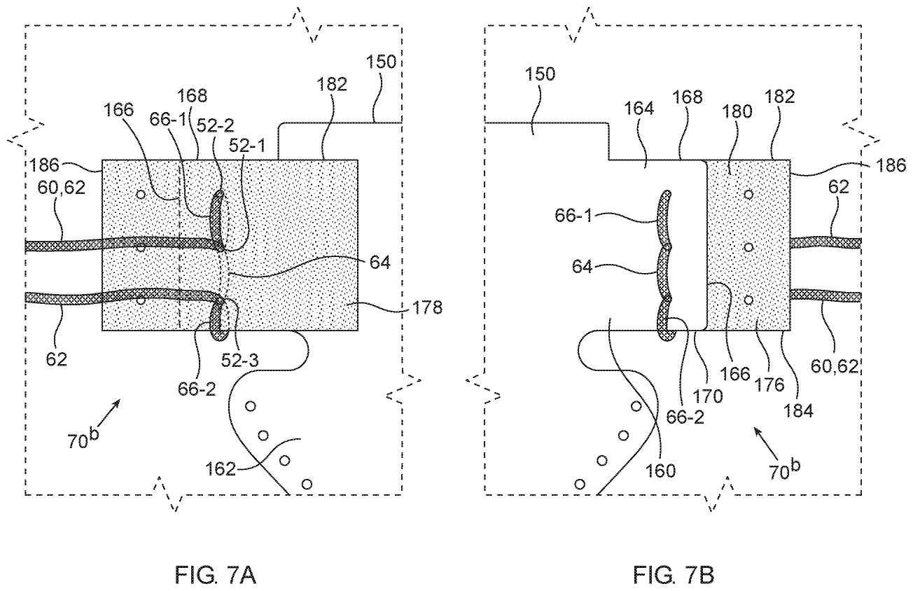

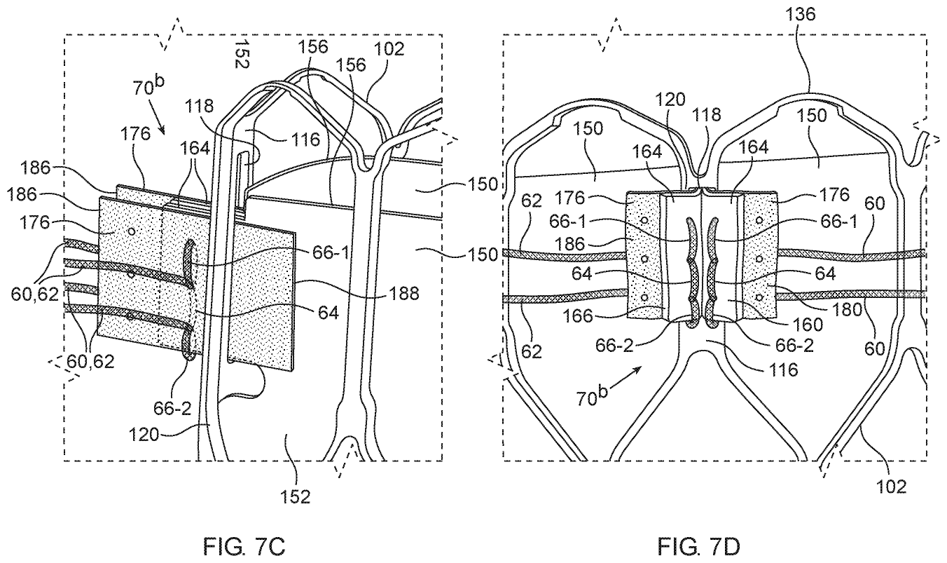

The tabs and reinforcement posts 176 attached thereto by a suture 60 following the stitching pattern 70^{a} shown in FIG. 6D, can be passed through a window opening 118 of a commissure support 116, from the internal side of the frame 102 to the external side (i.e., radially outwards), wherein the vertical stitches 64 along the second vertical stitch line 50b, as well as stitches bridging between vertical stitch lines, such as horizontal stitch 64-4, are passed through the window opening 118 while the folded portion 192a and its first vertical stitch line 50a remain radially inwards to the commissure support. The positions of the stitches 64 and the number of the stitches 64 of various stitching patterns disclosed herein, such as stitching pattern 70^{a} as well as other exemplary stitching patterns described below with respect to FIGS. 7A-17B, can be designed to provide narrower profiles of the portions passed through the window opening 118, allowing the window openings 118 of commissure supports 116 to have reduced profiles that can, in turn, reduces the overall crimp profile of the prosthetic valve.

Once the tabs 164 and reinforcement posts 176 secured thereto are radially passed through the window opening 118, additional stitches of the stitching pattern can be formed, for example during follow-up stages of commissure formation. A portion of the stitching pattern 70 that includes only stitches formed prior to the stage of passing the tabs 164 of adjacent leaflets 150 through the window opening 118, such as the stitches 64 of the stitching pattern 70^{a} shown in FIG. 6D, are referred to, herein, as a securement stitching pattern, as the stitches of this portion of the stitching pattern serve primarily to secure the reinforcement post 176 to the tab 164, while optionally, but not necessarily, also including stitches for securing overfolded layers of a first folded portion 192a to each other. A securement stitching pattern by which a reinforcement posts 176 is coupled to a respective tab 164 of a leaflet 150, can be formed off the frame of the prosthetic valve, for example in a flat configuration of the leaflet 150.

When tabs 164 of adjacent leaflets 150 are passed through a window opening 118 during an initial stage of commissure formation, such as the first stage illustrated in FIG. 5A, and prior to folding sideways any portions of the tabs 164 of reinforcement posts 176 or wrapping them around sidewalls 120, as exemplified in FIGS. 5B-5C for example, the first post section 194a is the section of the reinforcement tab that remains radially inwards to the commissure support 116, while the remainder of the reinforcement sections, including second post section 194b and an optional third post section 194c, are disposed radially outwards to the commissure support 116. Thus, a first folded portion 192a that can be pre-formed by overfolding the first post section 194a, resulting in a greater thickness of the two layers of the first post section 194a at this region, remains radially inwards without having to pass the greater thickness through the window opening 118.

The third post section 194c can be a section of the reinforcement post configured to form a second folded portion 192b, as shown for example in FIGS. 5B-5C, and can extend, at least partially, past the tab outer edge 166 prior to being overfolded. When a second folded portion 192b is formed, the overfolding occurs after the initial stage of passing the tabs 164 through the window opening 118 as shown in FIG. 5A for example. Thus, subsequent stitches passing through layers of the reinforcement post 176 to form a second folded portion 192b will not part of the securement stitching pattern.

A vertical stitch line extending along the first post section 194a, such as the first vertical stitch line 50a shown in FIG. 6D, is also referred to, herein, as an inner fold vertical stitch line, configured to pass through the two folded layers of the reinforcement post 176 to maintain the first post section 194a in a folded configuration, without passing through the leaflet 150. When tabs 164 of adjacent leaflets 150 are passed through a window opening 118 during an initial stage of commissure formation, such as the first stage illustrated in FIG. 5A, and prior to folding sideways any portions of the tabs 164 of reinforcement posts 176 or wrapping them around sidewalls 120, as exemplified in FIGS. 5B-5C for example, the inner fold vertical stitch line remains radially inwards to the commissure support 116.

A vertical stitch line extending along the second post section 194b, such as the second vertical stitch line 50b shown in FIG. 6D, is also referred to, herein, as a post securement vertical stitch line, configured to secure the reinforcement post 176 to the respective tab 164 prior to and during passing of the tabs 164 through the window opening 118. Thus, a securement vertical stitch line is necessarily formed as part of the securement stitching pattern 70, formed prior to passing of the tabs 164 through the window opening 118. When tabs 164 of adjacent leaflets 150 are passed through a window opening 118 during an initial stage of commissure formation, such as the first stage illustrated in FIG. 5A, and prior to folding sideways any portions of the tabs 164 of reinforcement posts 176 or wrapping them around sidewalls 120, as well as prior to forming additional subsequent stitches passing through the reinforcement posts 176 and/or the tab 164, as exemplified in FIGS. 5B-5C for example, the post securement vertical stitch line is positioned radially outwards to the commissure support 116.

When a securement stitching pattern 70 includes an inner fold vertical stitch line to form a first folded portion 192a, the securement stitching pattern 70 can further include, in some examples, at least one horizontal stitch, such as horizontal stitch 64-4 shown in FIG. 6D, extending between the inner fold vertical stitch line and the post securement vertical stitch line. In this manner, a single suture 60 is used to form a continuous securement stitching pattern 70 rather than forming two separated vertical stitch lines.

In some examples, the post securement vertical stitch line of a securement stitching pattern 70 does not extends along the entire height of the tab 164, between the outflow edge 168 and the inflow edge 170 of the tab 164. Similarly, in some examples, the post securement vertical stitch line of a securement stitching pattern 70 does not include loops 66 extending around both the outflow edge 168 and the inflow edge 170 of the tab 164.

For example, the vertical stitches 64-5 and 64-6 of along the post securement vertical stitch line 50b of the stitching pattern 70^{a}, shown in FIG. 6D, are axially offset from both the inflow edge 170 and the outflow edge 168 of the tab 164. Such configurations allow tissue material, which may be squeezed through a relatively narrow window opening 118, to expand or fold sideways to some extent along portions of the tabs 164 that do not include stitches 64, such as regions immediate the outflow edge 168 and/or the inflow edge 170 of the tab 164, facilitating easier passage of the tabs 164 through the commissure support 116.

As illustrated in FIGS. 6B and 6D, the inner fold vertical stitch line 50a of securement stitching pattern 70^{a} includes three vertical stitches 64, wherein the uppermost vertical stitch 64-1 is distally offset from the post outflow edge 182, and wherein the lowermost vertical stitch 64-3 is proximally offset from the post inflow edge 184. An uppermost vertical stitch 64 of a vertical stitch line 50 is defined as the vertical stitch 64 that is closest to the post outflow edge 182. Similarly, a lowermost vertical stitch 64 of a vertical stitch line 50 is defined as the vertical stitch 64 that is closest to the post inflow edge 184.

As further illustrated in FIG. 6D, the post securement vertical stitch line 50b of securement stitching pattern 70^{a} includes two vertical stitches 64, wherein the uppermost vertical stitch 64-6 is distally offset from the tab outflow edge 168, and wherein the lowermost vertical stitch 64-5 is proximally offset from the tab inflow edge 170. The securement stitching pattern 70^{a} further includes a single horizontal stitch 64-4 extending between the inner fold vertical stitch line 50a and the post securement vertical stitch line 50b. In some examples, as further illustrated in FIG. 6D, the securement stitching pattern 70^{a} can further include a single horizontal stitch 64-7 extending from the post securement vertical stitch line 50b in a direction towards the post outer edge 186.

FIGS. 7A and 7B show an exemplary securement stitching pattern 70^{b}, viewed from the outer side (facing the outer surface 162 of leaflet 150) and the inner side (facing the inner surface 160 of the leaflet) respectively. The securement stitching pattern 70^{b} shown in FIGS. 7A-7B is shown to include a single vertical stitch line 50 at the second post section 194b, which is the post securement vertical stitch line. In some examples, as illustrated in FIGS. 7A-7B, the first post section 194a is not necessarily folded over itself to form a first folded portion 192a, such that the securement stitching pattern 70^{b} can be devoid of an inner fold vertical stitch line.

The post securement vertical stitch line 50 of a securement stitching pattern 70^{b}, as illustrated in FIGS. 7A-7B, includes a first loop 66-1 extending between a first penetration point 52-1 and a second penetration point 52-2 along the first side 178 of the reinforcement post 176 and the inner surface 160 of the tab 164, a vertical stitch 64 extending between the second penetration point 52-2 and a third penetration point 52-3 along the inner surface 160 of the tab 164, and a second loop 66-2 extending through the third penetration point 52-3 along the first side 178 of the reinforcement post 176 and the inner surface 160 of the tab 164 and around the tab inflow edge 170.

The second penetration point 52-2 is distally offset from the tab outflow edge 168, the first penetration point 52-1 is distal to the second penetration point 52-2, and the third penetration point 52-3 is distal to the second penetration point 52-2 and is proximally offset from the tab inflow edge 170. As shown, the first loop 66-1, defining the uppermost portion of the suture 60 along the vertical stitch line 50, is axially offset from the tab outflow edge 168, and the vertical stitch 64 extends between the first loop 66-1 and the second loop 66-2. In other words, the post securement vertical stitch line 50 of securement stitching pattern 70^{b} includes two loops 66 and a single vertical stitch 64 extending between the two loops 66, wherein the uppermost loop 66-1 is distally offset from the tab outflow edge 168, and wherein the lowermost loop 66-2 extends around the tab inflow edge 170. Tails 62 of the suture 60 can extend from the first 52-1 and third 52-3 penetration points, for example along the first side 178 of the reinforcement post 176.

Loops 66 of securement stitching patterns 70 disclosed herein can be formed along post securement vertical stitch line 50. An uppermost loop 66 of a post securement vertical stitch line 50 is defined as the loop 66 that is closest to the tab outflow edge 168. Similarly, a lowermost loop 66 of a post securement vertical stitch line 50 is defined as the loop 66 that is closest to the tab inflow edge 170.

FIG. 7C shows tabs 164 of adjacent leaflets 150, along with reinforcement posts 176 secured thereto by the securement stitching pattern 70^{b} of FIGS. 7A-7B, passed through a window opening 118 of commissure support 116, such that the second post sections 194b along with the post securement vertical stitch line 50 formed therein are positioned radially outwards to the commissure support 116, while the first post section 194a remains positioned radially inwards to the commissure support 116. FIG. 7D shows the portions of the tabs 164 passed through the window opening 118 spread sideways, away from each other, exposing the stitches and loops of the respective post securement vertical stitch lines 50 along the inner surfaces 160 of the tabs 164, following the pattern shown in FIG. 7B for example.

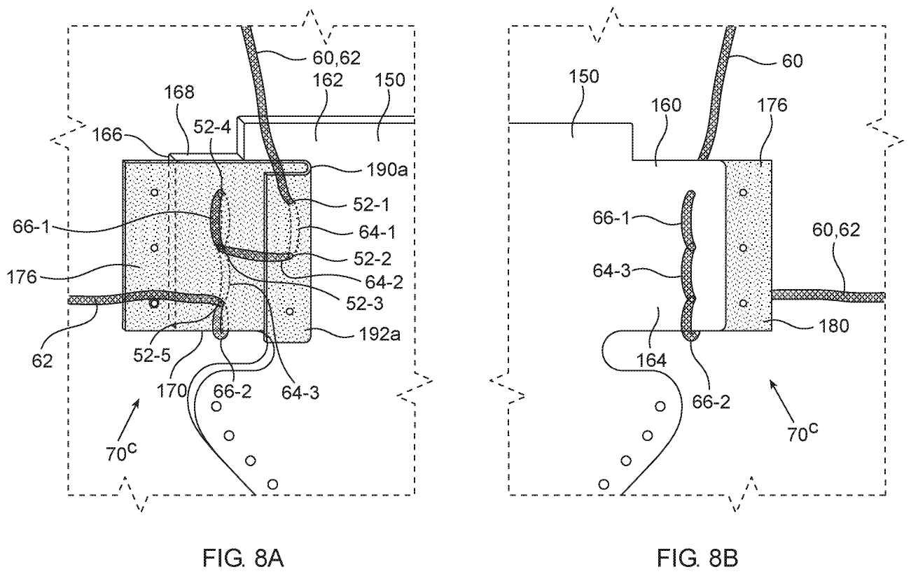

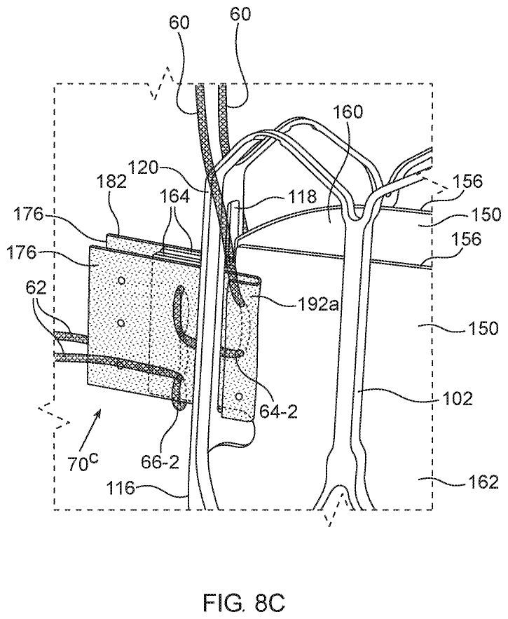

FIGS. 8A and 8B show an exemplary securement stitching pattern 70^{c}, viewed from the outer side and the inner side, respectively. The securement stitching pattern 70^{c} shown in FIGS. 8A-8B can have a post securement vertical stitch line 50b that can be generally similar to the post securement vertical stitch line of stitching pattern 70^{b} shown in FIGS. 7A-7D, except that the stitching pattern 70^{c} is further shown to include an inner fold vertical stitch line 50a configured to retain a first folded portion 192a of the reinforcement post 176 in an overfolded configuration.

An inner fold vertical stitch line 50a of a securement stitching pattern 70^{c}, illustrated in FIG. 8A, includes a first vertical stitch 64-1 extending over the inner surface of the folded portion 192a, between a first penetration point 52-1 and a second penetration point 52-2. A tail 62 of the suture 60 can extend from the first penetration point 52-1, for example along an outer surface of the folded portion 192a. The first penetration point 52-1 is distally offset from the post outflow edge 182, and the second penetration point 52-2 is distal to the first penetration point 52-1 and is proximally offset from the post inflow edge 184.

The securement stitching pattern 70^{c} further includes a second stitch 64-2, which is a horizontal stitch, extending between the second penetration point 52-2 of the inner fold vertical stitch line 50a and a third penetration point 52-3 of the post securement vertical stitch line 50b. The horizontal stitch 64-2 is disposed over outer surfaces of the reinforcement post 176, extending over part of the folded portion 192a, the post inner edge 188, and part of the second post section 194b.

The post securement vertical stitch line 50b of the securement stitching pattern 70^{c}, as illustrated in FIG. 8A-8B, includes a first loop 66-1 extending between the third penetration point 52-3 and a fourth penetration point 52-4 along the first side 178 of the reinforcement post 176 and the inner surface 160 of the tab 164, a third stitch 64-3, which is a vertical stitch, extending between the third penetration point 52-3 and a fifth penetration point 52-5 along the inner surface 160 of the tab 164, and a second loop 66-2 extending through the fifth penetration point 52-5 along the first side 178 of the reinforcement post 176 and the inner surface 160 of the tab 164 and around the tab inflow edge 170.

The fourth penetration point 52-4 is distally offset from the tab outflow edge 168, the third penetration point 52-3 is distal to the fourth penetration point 52-4, and the fifth penetration point 52-5 is distal to the third penetration point 52-3 and is proximally offset from the tab inflow edge 170. As shown, the first loop 66-1, defining the uppermost portion of the suture 60 along the post securement vertical stitch line 50b, is axially offset from the tab outflow edge 168, and the vertical stitch 64-3 extends between the first loop 66-1 and the second loop 66-2. A tail 62 of the suture 60 can extend from the fifth penetration point 52-5, for example along an outer surface of the reinforcement post 176.

FIG. 8C shows tabs 164 of adjacent leaflets 150, along with reinforcement posts 176 secured thereto by the securement stitching pattern 70^{c} of FIGS. 8A-8B, passed through a window opening 118 of commissure support 116, such that the second post section 194b along with the post securement vertical stitch line 50b formed therein is positioned radially outwards to the commissure support 116, while the first folded portion 192a along with the first vertical stitch line 50a extending therethrough remain positioned radially inwards to the commissure support 116.

As shown in FIGS. 8A-8C, the inner fold vertical stitch line 50a of securement stitching pattern 70^{c} includes a single vertical stitch 64-1 which is axially offset from both the post outflow edge 182 and post inflow edge 184, and can be closer to the post outflow edge 182 than the post inflow edge 184. The post securement vertical stitch line 50b of securement stitching pattern 70^{c} includes two loops 66 and a single vertical stitch 64-3 extending between the two loops 66, wherein the uppermost loop 66-1 is distally offset from the tab outflow edge 168, and wherein the lowermost loop 66-2 extends around the tab inflow edge 170. The securement stitching pattern 70^{c} further includes a single horizontal stitch 64-2 extending between the inner fold vertical stitch line 50a and the post securement vertical stitch line 50b.

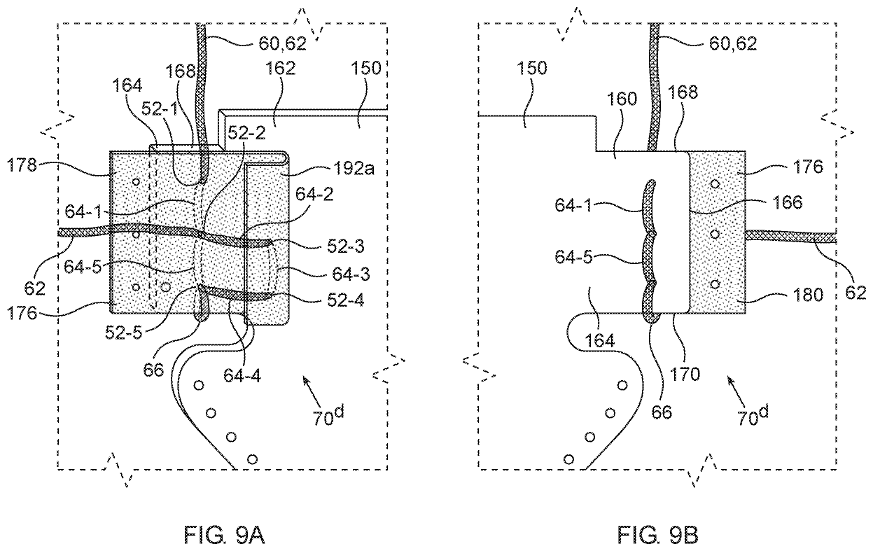

FIGS. 9A and 9B show an exemplary securement stitching pattern 70^{d}, viewed from the outer side and the inner side, respectively. A post securement vertical stitch line 50b of a securement stitching pattern 70^{d} includes a first vertical stitch 64-1 extending along the inner surface 160 of the tab 164, between a first penetration point 52-1 and a second penetration point 52-2, a fifth vertical stitch 64-5 extending along the inner surface 160 of the tab 164 between the second penetration point 52-2 and a fifth penetration point 52-5, and a loop 66 extending through the fifth penetration point 52-5 along the first side 178 of the reinforcement post 176 and the inner surface 160 of the tab 164 and around the tab inflow edge 170.

An inner fold vertical stitch line 50a of the securement stitching pattern 70^{d}, illustrated in FIG. 9A, includes a third vertical stitch 64-3 extending over the inner surface of the folded portion 192a, between a third penetration point 52-3 and a fourth penetration point 52-4. The securement stitching pattern 70^{d} further includes a second stitch 64-2, which is a horizontal stitch, extending between the second penetration point 52-2 of the post securement vertical stitch line 50b and a third penetration point 52-3 of the inner fold vertical stitch line 50a, and a fourth stitch 64-4, which is also a horizontal stitch, extending between the fourth penetration point 52-4 of the inner fold vertical stitch line 50a and a fifth penetration point 52-5 of the post securement vertical stitch line 50b. The horizontal stitches 64-2 and 64-4 are disposed over outer surfaces of the reinforcement post 176, extending over part of the folded portion 192a, the post inner edge 188, and part of the second post section 194b. Tails 62 of the suture 60 can extend from the first 52-1 and the second 52-2 penetration points, for example along an outer surface of the reinforcement post 176.

The first penetration point 52-1 is distally offset from the tab outflow edge 168, the second penetration point 52-2 is distal to the first penetration point 52-1, and the fifth penetration point 52-5 is distal to the second penetration point 52-2 and is proximally offset from the tab inflow edge 170. The third penetration point 52-3 is distally offset from the post outflow edge 182, and the fourth penetration point 52-4 is distal to the third penetration point 52-3 and is proximally offset from the post inflow edge 184.

As shown in FIGS. 9A-9B, the inner fold vertical stitch line 50a of securement stitching pattern 70^{d} includes a single vertical stitch 64-3 which is axially offset from both the post outflow edge 182 and post inflow edge 184, and can be closer to the post inflow edge 184 than the post outflow edge 182. The post securement vertical stitch line 50b of securement stitching pattern 70^{d} includes two subsequent vertical stitches 64 and a single loop 66, wherein the uppermost vertical stitch 64-1 is distally offset from the tab outflow edge 168, and wherein the loop 66 extends around the tab inflow edge 170. The securement stitching pattern 70^{d} further includes two horizontal stitch 64-2 and 64-4, each extending between the inner fold vertical stitch line 50a and the post securement vertical stitch line 50b, wherein both horizontal stitch 64-2 and 64-4 are parallel to each other and are axially spaced from each other.

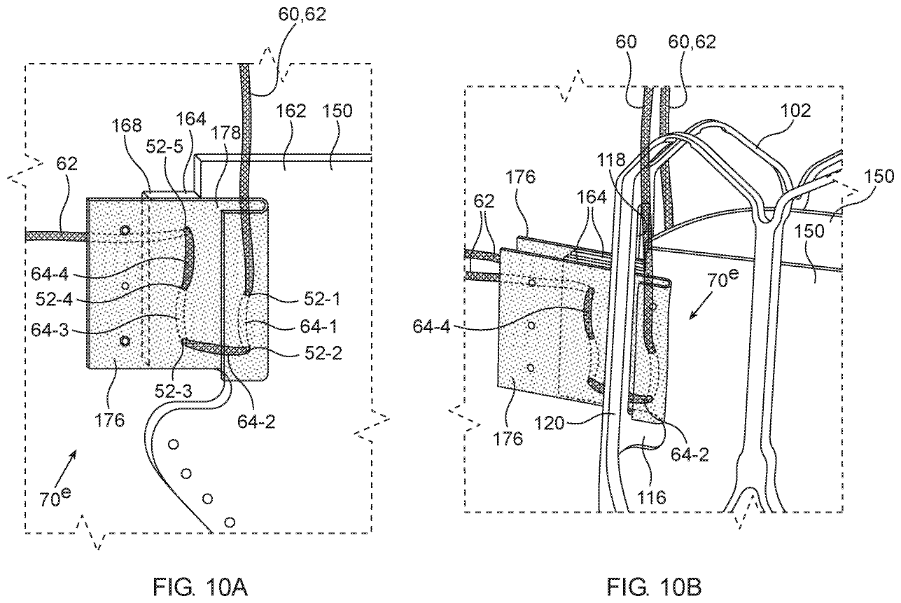

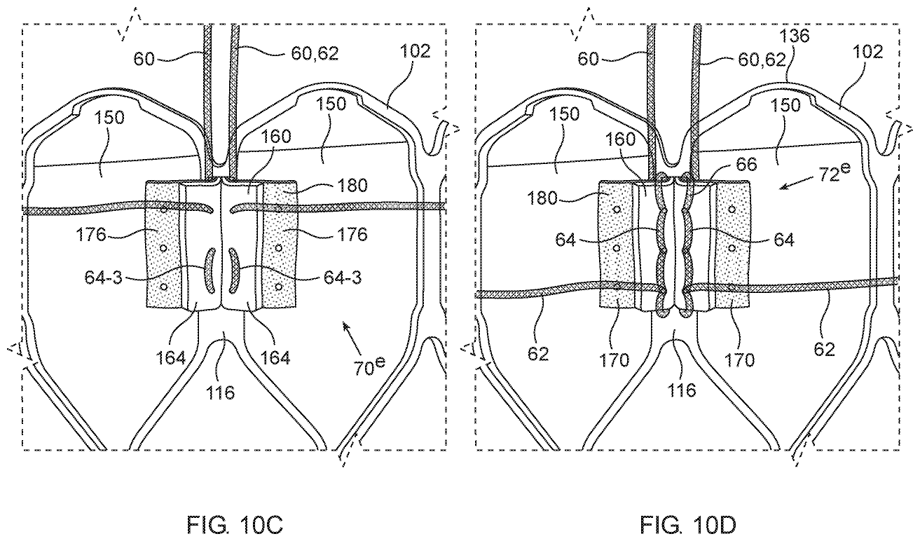

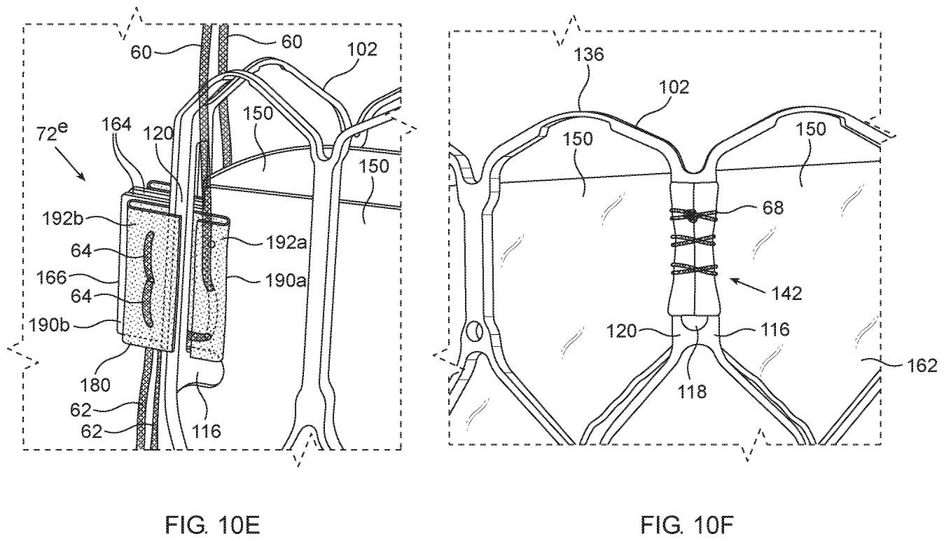

FIG. 10A shows an exemplary securement stitching pattern 70^{e}, viewed from the outer side. An inner fold vertical stitch line 50a of a securement stitching pattern 70^{e} includes a first vertical stitch 64-1 extending over the inner surface of the first folded portion 192a, between a first penetration point 52-1 and a second penetration point 52-2. A tail 62 of the suture 60 can extend from the first penetration point 52-1, for example along an outer surface of the folded portion 192a. The first penetration point 52-1 is distally offset from the post outflow edge 182, and the second penetration point 52-2 is distal to the first penetration point 52-1 and is proximally offset from the post inflow edge 184.

The securement stitching pattern 70^{e} further includes a second stitch 64-2, which is a horizontal stitch, extending between the second penetration point 52-2 of the inner fold vertical stitch line 50a and a third penetration point 52-3 of the post securement vertical stitch line 50b. The horizontal stitch 64-2 is disposed over outer surfaces of the reinforcement post 176, extending over part of the folded portion 192a, the post inner edge 188, and part of the second post section 194b.

The post securement vertical stitch line 50b of the securement stitching pattern 70^{e} includes a third vertical stitch 64-3 extending between the third penetration point 52-3 and a fourth penetration point 52-4 along the inner surface 160 of the tab 164, and a fourth vertical stitch 64-4 extending between the fourth penetration point 52-4 and a fifth penetration point 52-5 along the first side 178 of the reinforcement post 176. A tail 62 of the suture 60 can extend from the fifth penetration point 52-5, for example along the inner surface 160 of the tab 164. The third penetration point 52-3 is proximally offset from the tab inflow edge 170, the fourth penetration point 52-4 is proximal to the third penetration point 52-3, and the fifth penetration point 52-5 is proximal to the fourth penetration point 52-4.

As shown in FIG. 10A, the inner fold vertical stitch line 50a of securement stitching pattern 70^{e} includes a single vertical stitch 64-1 which is axially offset from both the post outflow edge 182 and post inflow edge 184, and can be closer to the post inflow edge 184 than the post outflow edge 182. The post securement vertical stitch line 50b of securement stitching pattern 70^{c} includes two subsequent vertical stitches 64, wherein the uppermost vertical stitch 64-4 is distally offset from the tab outflow edge 168, and wherein the lowermost vertical stitch 64-3 is proximally offset from the tab inflow edge 170. The securement stitching pattern 70^{e} further includes a single horizontal stitch 64-2 extending between the inner fold vertical stitch line 50a and the post securement vertical stitch line 50b.

FIG. 10B shows tabs 164 of adjacent leaflets 150, along with reinforcement posts 176 secured thereto by the securement stitching pattern 70^{e} of FIG. 10A, passed through a window opening 118 of commissure support 116, such that the second post sections 194b along with the post securement vertical stitch lines 50b formed therein are positioned radially outwards to the commissure support 116, while the first folded portion 192a remains positioned radially inwards to the commissure support 116. FIG. 10C shows the portions of the tabs 164 passed through the window opening 118 spread sideways, away from each other, exposing the stitches the respective post securement vertical stitch lines 50b along the inner surfaces 160 of the tabs 164.

FIG. 10D shows an optional following stage of forming a commissure 142 by which tails 62 of the sutures 60 can be passed in an in-and-out manner o form additional stitches of a complementary stitching pattern 72^{e} at the positions of the post securement vertical stitch lines 50b, resulting in vertical stitch lines 50 that can extend the entire height of the tabs 164, and optionally include loops 66 that extend around the outflow 168 and inflow 170 edges of the tabs 164. Thus, while post securement vertical stitch lines 50b of various exemplary securement stitching patterns 70 disclosed herein can include a limited number of stitches 64 and/or loops 66 sufficient to secure the reinforcement posts 176 to the tabs 164 during the act of passing the tabs 164 through the window opening 118, as shown for example in FIG. 10B, additional stitches 64 and/or loops 66 can be formed to improve attachment once the tabs 164 are passed through the window opening 118. This results in a final configuration that properly secures the reinforcement posts 176 to the tabs 164 with stitches and/or loops that can extend the entire height of the tabs 164, while a preliminary securement stitching pattern 70 includes a smaller number of stitches at regions that need to pass through the window opening 118, making it easier to pass the tabs 164 through narrower window openings 118 during initial stages of commissure formation.

FIG. 10E shows an optional following stage of forming a commissure 142 during which outer edge 188 of each reinforcement post 176 is folded outwardly (away from the tab 164) to form a second folded portion 192 b along a second fold line 190b. Depending on the length of the third post section 194c of the reinforcement post 176, the resulting second fold line 190b can be radially inwards to the tab outer edge 166, optionally exposing part of the tab 164 as illustrated in FIG. 10E for example, can be aligned with the tab outer edge 166, or can be radially outward to the tab outer edge 166.

One or more stitches and/or loops of the complementary stitching pattern 72^{e} can be then formed, for example by passing tails 62 of the sutures 60 in an in-and-out manner through the folded layers of the second folded portion 192, to retain it in a folded configuration. While two subsequent vertical stitches 64 are illustrated in FIG. 10E, it is to be understood that this is shown by way of illustration and not limitation, and that any other number of stitches and/or loops can be formed through a second folded portion 192b. In some examples, stitches 64 and/or loops 66 can extend through both layers of the second folded portion 192b without passing through the leaflet material of the corresponding tab 164, as described above for stitches of a first folded portion 192a. In some examples, stitches 64 and/or loops 66 can extend through both layers of the second folded portion 192b and through the corresponding tab 164.

Stitches 64 and/or loops 66 of a vertical stitch line 50c extending through the second folded portion 192b can extend through post pre-formed apertures 177 along the third post section 194, optionally defining a third vertical stitch line 50c. In some examples, the vertical stitch line formed through the second folded portion 192b is aligned with the post securement vertical stitch line, and stitches and/or loops of both vertical stitch lines can be aligned or even merged to form a coinciding vertical stitch line. In some examples, the vertical stitch line formed through the second folded portion 192b is offset from the post securement vertical stitch line.

FIG. 10F shows an optional commissure 142 formed after securing the tabs 164 to the sidewalls 120 of the commissure support 116. In some examples, one or more knots 68 can be formed around the tabs 164 and/or reinforcement posts 176 secured thereto. In some examples, formation of the commissure 142 can include stages similar to those described above with respect to FIGS. 5A-5C. In some examples, a wedge member 148 can be positioned between both tabs 164 of a commissure 142, radially outwards to the window opening 118.

While optional stages of commissure formation are described above and illustrated in FIGS. 10B-10F with respect to reinforcement posts 176 secured to tabs 164 by a stitching pattern 70^{e}, it is to be understood that this is not meant to be limiting, and that any exemplary stage of forming a commissure 142 secured to the commissure support 116 can be similarly implemented with respect to any other exemplary securement configuration disclosed herein, including with respect of any of the securement stitch patterns 70^{a}, 70^{b}, 70^{c}, or 70^{d} described above, or any of the securement stitch patterns 70^{f}, 70^{g}, 70^{h}, 70ⁱ, 70^{j} or 70^{m} described below.

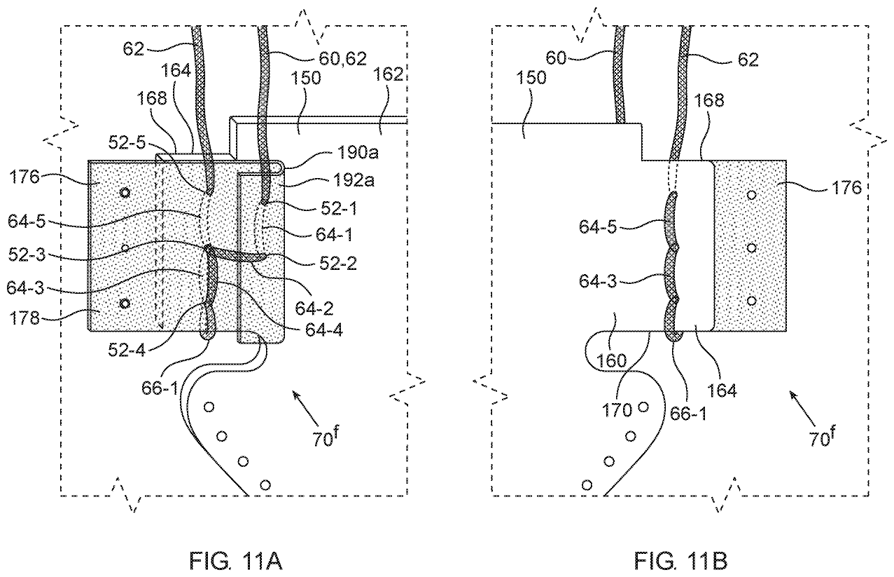

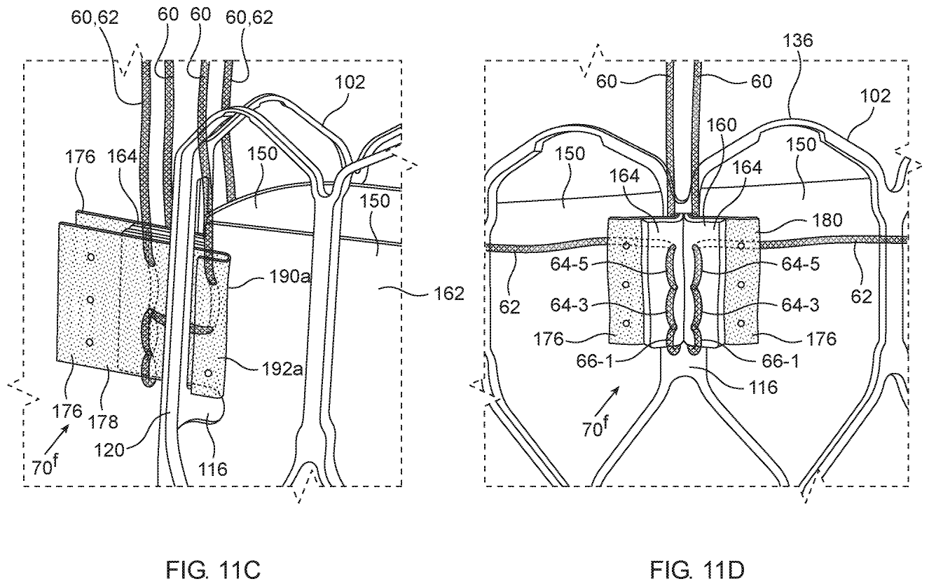

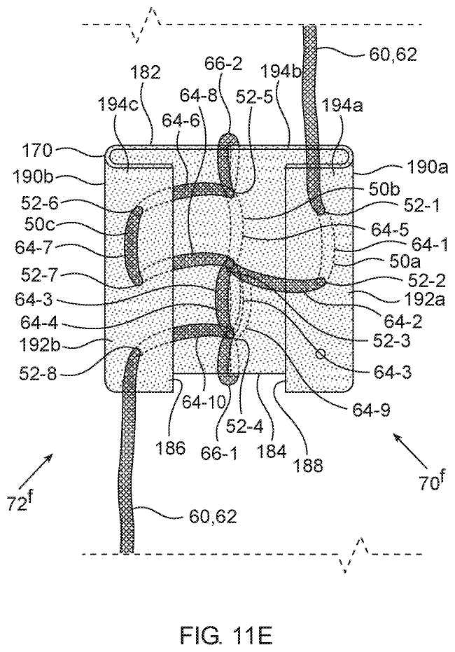

FIGS. 11A and 11B show an exemplary securement stitching pattern 70^{f}, viewed from the outer side and the inner side, respectively. An inner fold vertical stitch line 50a of a securement stitching pattern 70^{f} includes a first vertical stitch 64-1 extending over the inner surface of the folded portion 192a, between a first penetration point 52-1 and a second penetration point 52-2. A tail 62 of the suture 60 can extend from the first penetration point 52-1, for example along an outer surface of the folded portion 192a. The first penetration point 52-1 is distally offset from the post outflow edge 182, and the second penetration point 52-2 is distal to the first penetration point 52-1 and is proximally offset from the post inflow edge 184.

The securement stitching pattern 70^{f} further includes a second stitch 64-2, which is a horizontal stitch, extending between the second penetration point 52-2 of the inner fold vertical stitch line 50a and a third penetration point 52-3 of the post securement vertical stitch line 50b. The horizontal stitch 64-2 is disposed over outer surfaces of the reinforcement post 176, extending over part of the folded portion 192a, the post inner edge 188, and part of the second post section 194b.

The post securement vertical stitch line 50b of the securement stitching pattern 70^{f} includes a third vertical stitch 64-3 extending between the third penetration point 52-3 and a fourth penetration point 52-4 along the inner surface 160 of the tab 164, a first loop 66-1 extending through the fourth penetration point 52-4 along the first side 178 of the reinforcement post 176 and the inner surface 160 of the tab 164 and around the tab inflow edge 170, a fourth vertical stitch 64-4 extending between the fourth penetration point 52-4 and the third penetration point 52-3 along the first side 178 of the reinforcement post 176, and a fifth vertical stitch 64-5 extending between the third penetration point 52-3 and a fifth penetration point 52-5 along the inner surface 160 of the tab 164. A tail 62 of the suture 60 can extend from the fifth penetration point 52-5, for example along an outer surface of the reinforcement post 176.

The fifth penetration point 52-5 is distally offset from the tab outflow edge 168, the third penetration point 52-3 is distal to the fifth penetration point 52-5, and the fourth penetration point 52-4 is distal to the third penetration point 52-3 and is proximally offset from the tab inflow edge 170.

As shown in FIGS. 11A-11B, the inner fold vertical stitch line 50a of securement stitching pattern 70^{f} includes a single vertical stitch 64-1 which is axially offset from both the post outflow edge 182 and post inflow edge 184, and can be closer to the post outflow edge 182 than the post inflow edge 184. The post securement vertical stitch line 50b of securement stitching pattern 70^{f} includes three subsequent vertical stitches 64 and a loop 66, wherein the uppermost vertical stitch 64-5 is distally offset from the tab outflow edge 168, the two remaining vertical stitches 64-3 and 64-4 are aligned with each other and extend between the same penetration points 52-3 and 52-4 over opposite sides of the tab 164 and reinforcement post 176, and wherein the loop 66-1 is distal to all vertical stitches 64-3, 64-4, 64-5 and loop 66-2 extends around the tab inflow edge 170. The securement stitching pattern 70^{f} further includes a single horizontal stitch 64-2 extending between the inner fold vertical stitch line 50a and the post securement vertical stitch line 50b.

FIG. 11C shows tabs 164 of adjacent leaflets 150, along with reinforcement posts 176 secured thereto by the securement stitching pattern 70^{f} of FIGS. 11A-11B, passed through a window opening 118 of commissure support 116, such that the second post sections 194b along with the post securement vertical stitch lines 50b formed therein are positioned radially outwards to the commissure support 116, while the first folded portion 192a remains positioned radially inwards to the commissure support 116. FIG. 11D shows the portions of the tabs 164 passed through the window opening 118 spread sideways, away from each other, exposing the stitches the respective post securement vertical stitch lines 50b along the inner surfaces 160 of the tabs 164.

FIG. 11E shows an optional continuation of the stitching configuration, during which the outer edge 188 of each reinforcement post 176 is folded outwardly (away from the tab 164) to form a second folded portion 192b along a second fold line 190b, and a third vertical stitch line 50c, which can be also referred to, herein, as an outer fold vertical stitch line, is formed. The outer fold vertical stitch line 50c is configured to pass through the two folded layers of the reinforcement post 176 to maintain the third post section 194c in a folded configuration. The outer fold vertical stitch line is formed after adjacent leaflets 150 are passed through a window opening 118, and is thus positioned radially outward from the commissure support 116.

As mentioned above with respect to FIG. 10D for example, once the tabs 164 are passed through the window opening 118, additional stitches or loops can be added to the post securement vertical stitch line 50b, to provide improved securement of the reinforcement post 176 to the tab 164 along the entire height of the tab 164. As shown in FIG. 11E, once the tabs 164 are passed through the window openings, a second loop 66-2 can be added to the post securement vertical stitch line 50b of stitching pattern 70^{f}, extending through the fifth penetration point 52-5 along the first side 178 of the reinforcement post 176 and the inner surface 160 of the tab 164 and around the tab outflow edge 168.

It is to be understood that stitches or loops formed along the second 194b and/or third 194c sections of the reinforcement post 176 after passing the tabs 164 through the window opening 118 (i.e., stitches and/or loops extending through portions of the reinforcement posts 176 which are radially outward to the commissure support 116) are not considered part of the securement stitching pattern 70, but rather are part of a complementary stitching pattern 72 formed subsequently to the securement stitching pattern 70.

A complementary stitching pattern 72^{f} includes an uppermost loop 66-2 of the post securement vertical stitch line 50b, which is proximal to all stitches and the loop of the securement stitching pattern 70^{f} of the post securement vertical stitch line 50b. An additional ninth vertical stitch 64-9 of the complementary stitching pattern 72^{f} is added to the post securement vertical stitch line 50b over the inner surface 160 of the tab 164, between the third penetration point 52-3 and the fourth penetration point 52-4, such the ninth vertical stitch 64-9 is congruent with the third vertical stitch 64-3.

An outer fold vertical stitch line 50c of a complementary stitching pattern 72^{f} includes a seventh vertical stitch 64-7 extending over an outer surface of the second folded portion 192b, between a sixth penetration point 52-6 and a seventh penetration point 52-7.

The complementary stitching pattern 72^{f} further includes a sixth stitch 64-6, an eight stitch 64-8, and a tenth stitch 64-10, all of which are horizontal stitches disposed over outer surfaces of the reinforcement post 176, each extending over the first side 178 of the reinforcement member 176, and between the layers of the second folded portion 192 along parts of the stitches extending past the post outer edge 186. The sixth stitch 64-6 extends between the fifth penetration point 52-5 and the sixth penetration point 52-6, the eight stitch 64-8 extends between the seventh penetration point 52-7 and the third penetration point 52-3, and the tenth stitch 64-10 extends between the fourth penetration point 52-4 and an eighth penetration point 52-8. A tail 62 of the suture 60 can extend from the eighth penetration point 52-8, for example along an outer surface of the second folded portion 192b.

The sixth penetration point 52-6 is distally offset from the post outflow edge 182, the seventh penetration point 52-7 is distal to the sixth penetration point 52-6, and the eighth penetration point 52-8 is distal to the sixth penetration point 52-6 and is proximally offset from the post inflow edge 184.

As shown in FIG. 11E, the outer fold vertical stitch line 50c of complementary stitching pattern 72^{f} includes a single vertical stitch 64-7 which is axially offset from both the post outflow edge 182 and post inflow edge 184, and can be closer to the post outflow edge 182 than the post inflow edge 184. The complementary stitching pattern 72^{f} further includes three horizontal stitch 64-6, 64-8 and 64-10, which are parallel to each other and are axially spaced from each other.

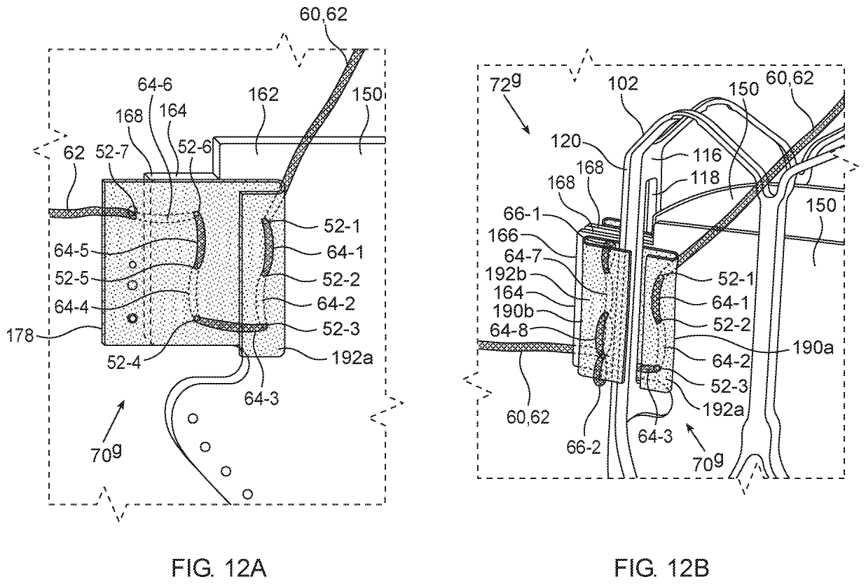

FIG. 12A shows an exemplary securement stitching pattern 70^{g}, viewed from the outer side. An inner fold vertical stitch line 50a of a securement stitching pattern 70^{g} includes a first vertical stitch 64-1 extending over an outer surface of the first folded portion 192a, between a first penetration point 52-1 and a second penetration point 52-2, and a second vertical stitch 64-2 extending over an inner surface of the first folded portion 192a, between the second penetration point 52-2 and a third penetration point 52-3. A tail 62 of the suture 60 can extend from the first penetration point 52-1, for example along the inner surface of the folded portion 192a. The first penetration point 52-1 is distally offset from the post outflow edge 182, the second penetration point 52-2 is distal to the first penetration point 52-1, and the third penetration point 52-3 is distal to the second penetration point 52-2 and is proximally offset from the post inflow edge 184.

The securement stitching pattern 70^{g} further includes a third stitch 64-3, which is a horizontal stitch, extending between the third penetration point 52-3 of the inner fold vertical stitch line 50a and a fourth penetration point 52-4 of the post securement vertical stitch line 50b. The horizontal stitch 64-3 is disposed over outer surfaces of the reinforcement post 176, extending over part of the first folded portion 192a, the post inner edge 188, and part of the second post section 194b.

The post securement vertical stitch line 50b of the securement stitching pattern 70^{g} includes a fourth vertical stitch 64-4 extending between the fourth penetration point 52-4 and a fifth penetration point 52-5 along the inner surface 160 of the tab 164, and a fifth vertical stitch 64-5 extending between the fifth penetration point 52-5 and a sixth penetration point 52-6 along the first side 178 of the reinforcement post 176. The sixth penetration point 52-6 is distally offset from the tab outflow edge 168, the fifth penetration point 52-5 is distal to the sixth penetration point 52-6, and the fourth penetration point 52-4 is distal to the fifth penetration point 52-5 and is proximally offset from the tab inflow edge 170.

The securement stitching pattern 70^{g} can further include a sixth stitch 64-6, which is a horizontal stitch, extending between the sixth penetration point 52-6 of the post securement vertical stitch line 50b, and a seventh penetration point 52-7 formed in the third post section 194c. The seventh penetration point 52-7 can be part of a later formed outer fold vertical stitch line 50c, such that the horizontal stitch 64-6 can bridge between the post securement vertical stitch line 50b and outer fold vertical stitch line 50c. The horizontal stitch 64-6 can be disposed over the inner surface 160 of the tab 164, the tab outer edge 162, and an inner surface or second side 180 of reinforcement post 176, such as along part of the third post section 194c. A tail 62 of the suture 60 can extend from the seventh penetration point 52-7, for example along the first side 178 of the reinforcement post 176.

As shown in FIG. 12A, the inner fold vertical stitch line 50a of securement stitching pattern 70^{g} includes two subsequent vertical stitches 64, wherein the uppermost vertical stitch 64-1 is distally offset from the post outflow edge 182, and the lowermost vertical stitch 64-2 is proximally offset from the post inflow edge 184. The post securement vertical stitch line 50b of securement stitching pattern 70^{g} includes two subsequent vertical stitches 64, wherein the uppermost vertical stitch 64-5 is distally offset from the tab outflow edge 168, and wherein the lowermost vertical stitch 64-4 is proximally offset from the tab inflow edge 170. The securement stitching pattern 70^{g} further includes a single horizontal stitch 64-2 extending between the inner fold vertical stitch line 50a and the post securement vertical stitch line 50b, and can include a single horizontal stitch 64-6 extending from the post securement vertical stitch line 50b in a direction towards the post outer edge 186.

FIG. 12B shows a complementary stitching pattern 72^{g} formed after extending the tabs 164 of adjacent leaflets 150, along with reinforcement posts 176 secured thereto by the securement stitching pattern 70^{f} of FIG. 12A, and overfolding the third post sections 194 to form second folded portion 192b radially outward to the commissure support 116. The outer fold vertical stitch line 50c of complementary stitching pattern 72^{g} includes two loops 66-1 and 66-2, and two subsequent vertical stitches 64-7 and 64-8 disposed between the loops 66-1 and 66-2. The uppermost loop 66-1 extends around the post outflow edge 182, and the lowermost loop 66-2 extends around the post inflow edge 184. The uppermost vertical stitch 64-7 is distal to the uppermost loop 66-1, and can extend over an inner surface of the second folded portion 192b. The lowermost vertical stitch 64-8 is distal to the uppermost vertical stitch 64-7 and proximal to the lowermost loop 66-2, and can extend over an outer surface of the second folded portion 192b.

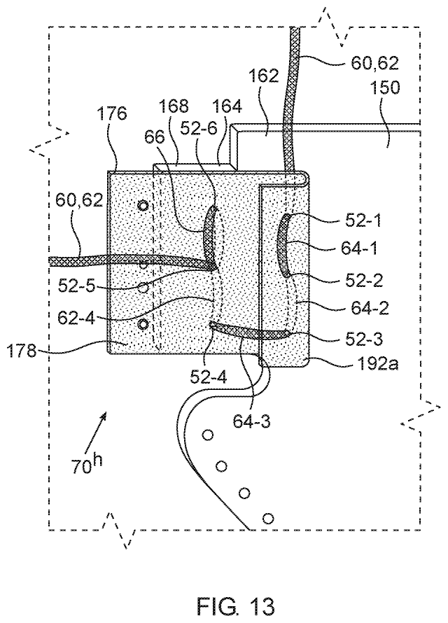

FIG. 13 shows an exemplary securement stitching pattern 70^{h}, viewed from the outer side. An inner fold vertical stitch line 50a of a securement stitching pattern 70^{h} includes a first vertical stitch 64-1 extending over an outer surface of the first folded portion 192a, between a first penetration point 52-1 and a second penetration point 52-2, and a second vertical stitch 64-2 extending over an inner surface of the first folded portion 192a, between the second penetration point 52-2 and a third penetration point 52-3. A tail 62 of the suture 60 can extend from the first penetration point 52-1, for example along the inner surface of the folded portion 192a. The first penetration point 52-1 is distally offset from the post outflow edge 182, the second penetration point 52-2 is distal to the first penetration point 52-1, and the third penetration point 52-3 is distal to the second penetration point 52-2 and is proximally offset from the post inflow edge 184.

The securement stitching pattern 70^{h} further includes a third stitch 64-3, which is a horizontal stitch, extending between the third penetration point 52-3 of the inner fold vertical stitch line 50a and a fourth penetration point 52-4 of the post securement vertical stitch line 50b. The horizontal stitch 64-3 is disposed over outer surfaces of the reinforcement post 176, extending over part of the first folded portion 192a, the post inner edge 188, and part of the second post section 194b.

The post securement vertical stitch line 50b of the securement stitching pattern 70^{h} includes a fourth vertical stitch 64-4 extending between the fourth penetration point 52-4 and a fifth penetration point 52-5 along the inner surface 160 of the tab 164, and a loop 66 extending between the fifth penetration point 52-5 and a sixth penetration point 52-6 along the first side 178 of the reinforcement post 176 and the inner surface 160 of the tab 164. The sixth penetration point 52-6 is distally offset from the tab outflow edge 168, the fifth penetration point 52-5 is distal to the sixth penetration point 52-6 and is proximally offset from the tab inflow edge 170. A tail 62 of the suture 60 can extend from the fifth penetration point 52-5, for example along the first side 178 of the reinforcement post 176.

As shown in FIG. 13, the inner fold vertical stitch line 50a of securement stitching pattern 70^{h} includes two subsequent vertical stitches 64, wherein the uppermost vertical stitch 64-1 is distally offset from the post outflow edge 182, and the lowermost vertical stitch 64-2 is proximally offset from the post inflow edge 184. The post securement vertical stitch line 50b of securement stitching pattern 70^{h} includes a single vertical stitch line 62-4 which is proximally offset from the tab inflow edge 170, and a single loop 66 which is distally offset from the tab outflow edge 168. The securement stitching pattern 70^{h} further includes a single horizontal stitch 64-2 extending between the inner fold vertical stitch line 50a and the post securement vertical stitch line 50b.

FIGS. 11A and 11B show an exemplary securement stitching pattern 70ⁱ, viewed from the outer side and the inner side, respectively. Securement stitching pattern 70ⁱ may be different from other securement stitching patterns disclosed herein in that the post securement vertical stitch line 50b of the securement stitching pattern 70ⁱ extends along the entire height of the tab 164, and includes loops 66 extending around both the outflow 168 and inflow 170 edges of the tab 164. Thus, utilization of securement stitching pattern 70ⁱ may be better suited for commissure supports 116 having slightly larger window openings 118. Nevertheless, similar to other securement stitching patterns 70 that include an inner fold vertical stitch line 50a, the securement stitching pattern 70ⁱ comprises a horizontal stitch 64 bridging between the inner fold vertical stitch line 50a and the post securement vertical stitch line 50b, such that both can be continuously formed from a single suture 60 instead of being separately formed.

An inner fold vertical stitch line 50a of a securement stitching pattern 70ⁱ includes a first vertical stitch 64-1 extending over the inner surface of the first folded portion 192a, between a first penetration point 52-1 and a second penetration point 52-2. A tail 62 of the suture 60 can extend from the first penetration point 52-1, for example along an outer surface of the folded portion 192a. The first penetration point 52-1 is proximally offset from the post inflow edge 184, and the second penetration point 52-2 is proximal to the first penetration point 52-1 and is distally offset from the post outflow edge 182.

The securement stitching pattern 70ⁱ further includes a second stitch 64-2, which is a horizontal stitch, extending between the second penetration point 52-2 of the inner fold vertical stitch line 50a and a third penetration point 52-3 of the post securement vertical stitch line 50b. The horizontal stitch 64-2 is disposed over outer surfaces of the reinforcement post 176, extending over part of the folded portion 192a, the post inner edge 188, and part of the second post section 194b.

The post securement vertical stitch line 50b of the securement stitching pattern 70ⁱ includes a third vertical stitch 64-3 extending between the third penetration point 52-3 and a fourth penetration point 52-4 along the inner surface 160 of the tab 164, a first loop 66-1 extending through the fourth penetration point 52-4 along the first side 178 of the reinforcement post 176 and the inner surface 160 of the tab 164 and around the tab outflow edge 168, a fourth vertical stitch 64-4 extending between the fourth penetration point 52-4 and the third penetration point 52-3 along the first side 178 of the reinforcement post 176, a fifth vertical stitch 64-5 extending between the third penetration point 52-3 and a fifth penetration point 52-5 along the inner surface 160 of the tab 164, and a second loop 66-2 extending through the fifth penetration point 52-5 along the first side 178 of the reinforcement post 176 and the inner surface 160 of the tab 164 and around the tab inflow edge 170. The fourth penetration point 52-4 is distally offset from the tab outflow edge 168, the third penetration point 52-3 is distal to the fourth penetration point 52-4, and the fifth penetration point 52-5 is distal to the third penetration point 52-3 and is proximally offset from the tab inflow edge 170

The securement stitching pattern 70ⁱ can further include a sixth stitch 64-6, which is a horizontal stitch, extending between the fifth penetration point 52-5 of the post securement vertical stitch line 50b, and a sixth penetration point 52-6 formed in the third post section 194c. The sixth penetration point 52-6 can be part of a later formed outer fold vertical stitch line, such that the horizontal stitch 64-6 can bridge between the post securement vertical stitch line 50b and outer fold vertical stitch line. The horizontal stitch 64-6 can be disposed over the first side 178 of the reinforcement post 176. A tail 62 of the suture 60 can extend from the sixth penetration point 52-6, for example along the second side 180 of the reinforcement post 176.

As shown in FIGS. 10A-10B, the inner fold vertical stitch line 50a of securement stitching pattern 70ⁱ includes a single vertical stitch 64-1 which is axially offset from both the post outflow edge 182 and post inflow edge 184, and can be closer to the post inflow edge 184 than the post outflow edge 182. The post securement vertical stitch line 50b of securement stitching pattern 70ⁱ includes two loops 66 and three vertical stitches 64, wherein the uppermost loop 66-1 extends around the tab outflow edge 168, the lowermost loop 66-2 extends around the tab inflow edge 170, the lowermost vertical stitch 64-5 is proximal to the lowermost loop 66-2, and the two remaining vertical stitches 64-3 and 64-4 extend between the uppermost loop 66-1 and the lowermost vertical stitch 64-5, passing through the same penetration point 52-3 and 52-4 over opposite sides of the tab 164 and reinforcement post 176. The securement stitching pattern 70ⁱ further includes a single horizontal stitch 64-2 extending between the inner fold vertical stitch line 50a and the post securement vertical stitch line 50b, and can include a single horizontal stitch 64-6 extending from the post securement vertical stitch line 50b in a direction towards the post outer edge 186.

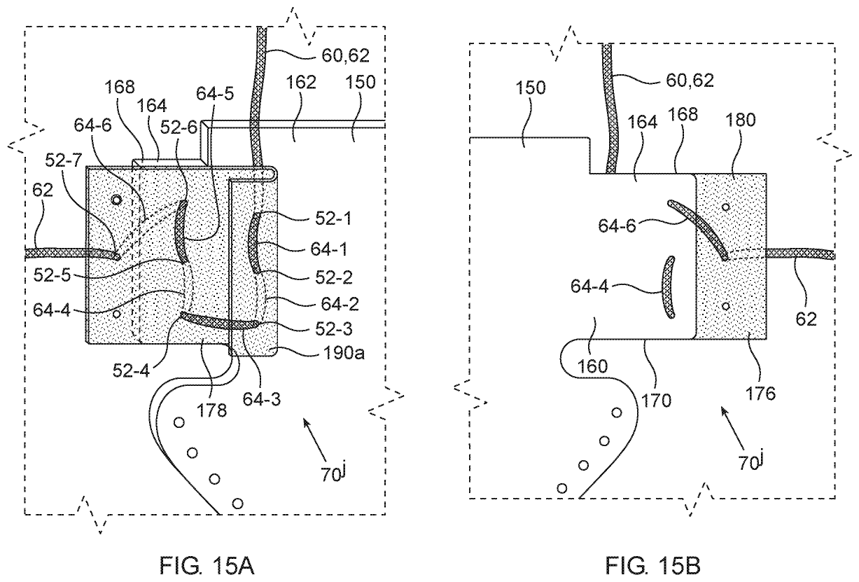

FIGS. 15A and 15B show an exemplary securement stitching pattern 70^{j}, viewed from the outer side and the inner side, respectively. An inner fold vertical stitch line 50a of a securement stitching pattern 70^{j} includes a first vertical stitch 64-1 extending over an outer surface of the first folded portion 192a, between a first penetration point 52-1 and a second penetration point 52-2, and a second vertical stitch 64-2 extending over an inner surface of the first folded portion 192a, between the second penetration point 52-2 and a third penetration point 52-3. A tail 62 of the suture 60 can extend from the first penetration point 52-1, for example along the inner surface of the folded portion 192a. The first penetration point 52-1 is distally offset from the post outflow edge 182, the second penetration point 52-2 is distal to the first penetration point 52-1, and the third penetration point 52-3 is distal to the second penetration point 52-2 and is proximally offset from the post inflow edge 184.

The securement stitching pattern 70^{j} further includes a third stitch 64-3, which is a horizontal stitch, extending between the third penetration point 52-3 of the inner fold vertical stitch line 50a and a fourth penetration point 52-4 of the post securement vertical stitch line 50b. The horizontal stitch 64-3 is disposed over outer surfaces of the reinforcement post 176, extending over part of the first folded portion 192a, the post inner edge 188, and part of the second post section 194b.

The post securement vertical stitch line 50b of the securement stitching pattern 70^{j} includes a fourth vertical stitch 64-4 extending between the fourth penetration point 52-4 and a fifth penetration point 52-5 along the inner surface 160 of the tab 164, and a fifth vertical stitch 64-5 extending between the fifth penetration point 52-5 and a sixth penetration point 52-6 along the first side 178 of the reinforcement post 176. The sixth penetration point 52-6 is distally offset from the tab outflow edge 168, the fifth penetration point 52-5 is distal to the sixth penetration point 52-6, and the fourth penetration point 52-4 is distal to the fifth penetration point 52-5 and is proximally offset from the tab inflow edge 170.

The securement stitching pattern 70^{j} can further include a sixth stitch 64-6, which is a diagonal stitch, extending between the sixth penetration point 52-6 of the post securement vertical stitch line 50b, and a seventh penetration point 52-7 formed in the third post section 194c. The seventh penetration point 52-7 can be part of a later formed outer fold vertical stitch line, such that the horizontal stitch 64-6 can bridge between the post securement vertical stitch line 50b and outer fold vertical stitch line. The diagonal stitch 64-6 can be disposed over the inner surface 160 of the tab 164, the tab outer edge 162, and an inner surface or second side 180 of reinforcement post 176, such as along part of the third post section 194c. A tail 62 of the suture 60 can extend from the seventh penetration point 52-7, for example along the first side 178 of the reinforcement post 176.

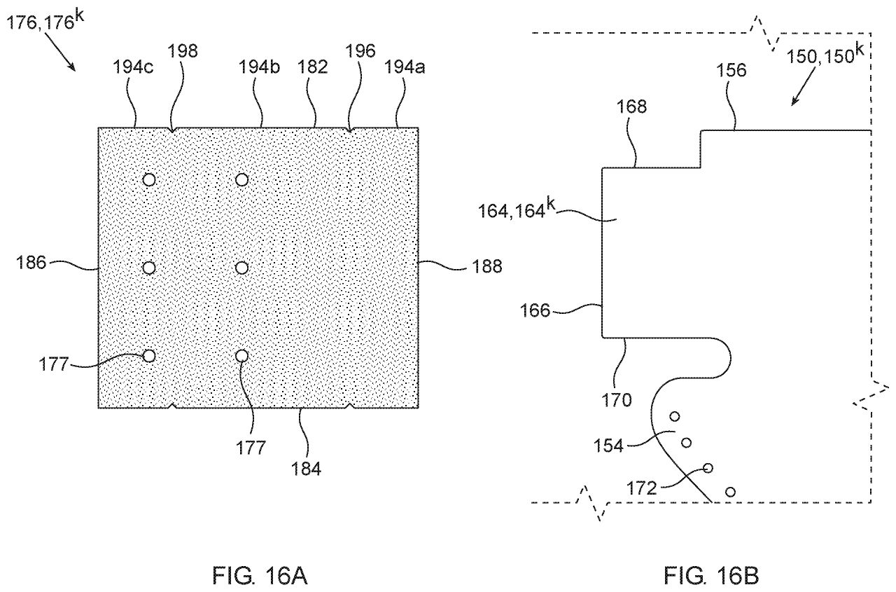

FIG. 16A is a front view in elevation of an exemplary reinforcement post 176^{k}, which is an exemplary implementation of reinforcement post 176, and thus can include any of the features described for reinforcement post 176 throughout the current disclosure, except that the reinforcement post 176^{k} can further include one or more notches configured to provide a visual marking of regions of interest of the reinforcement post 176^{k} during the assembly procedure. In some examples, a reinforcement post 176 includes one or more inner notches 196 at a position along which the reinforcement post 176 is to be folded to form an inner or first folded portion 192. Thus, inner notches 196 are formed at a position of the first fold line 190. While two inner notches 196 are illustrated in FIG. 16A for reinforcement post 176^{k}, one at each the outflow 182 and inflow 184 edges of the reinforcement post 176, it is to be understood that in some examples, a single inner notch 196 can be formed either at the post outflow edge 182 or at the post inflow edge 184.

In some examples, a reinforcement post 176 includes one or more outer notches 198 that mark the desired position of the tab outer edge 166. Thus, a reinforcement member 176 can be conveniently placed over a corresponding tab 164 during the assembly position at the correct position relative to the tab 164, by aligning the outer notches 198 with the tab outer edge 166. While two outer notches 198 are illustrated in FIG. 16A for reinforcement post 176^{k}, one at each the outflow 182 and inflow 184 edges of the reinforcement post 176, it is to be understood that in some examples, a single outer notch 198 can be formed either at the post outflow edge 182 or at the post inflow edge 184.

While the exemplary reinforcement post 176^{k} is shown in FIG. 16a to include both inner notches 196 and outer notches 198, it is to be understood that in some examples, a reinforcement post 176 can include one or more inner notches 196 without outer notches 198, or one or more outer notches 198 without inner notches 196.

In some examples, a reinforcement post that includes inner 196 and/or outer 198 notches, such as reinforcement post 176^{k}, can further include post pre-formed apertures 177 designating locations of penetration points 52 through which one or more vertical stitch lines 50 can be formed, as described above with respect to reinforcement post 176^{a}, for example.

FIG. 16B is a front view in elevation of a portion of an exemplary leaflet 150^{k}, which is similar to any example described herein for leaflet 150, except that in contrast to exemplary leaflet 150^{a} described above with respect to FIG. 6C, the tab 164^{k} of leaflet 150^{k} can be devoid of tab pre-formed apertures 174. A reinforcement post 176 that includes pre-formed apertures 177 and one or more outer notches 198, such as reinforcement post 176^{k}, can be placed over a leaflet 150^{k} devoid of pre-formed apertures 174, wherein the outer notches 198 can assist in proper positioning of the reinforcement post 176^{k} relative to the tab 164^{k}, and the post pre-formed apertures 177 can mark the penetration points 52 through which a suture should be passed when to form a desired stitching pattern, thus obviating the need to pre-form additional apertures 174 through the tab 164, advantageously simplifying production and optionally leading to cost reductions.

While exemplary reinforcement post 176^{k} is illustrated in FIG. 16A to include two vertical columns of apertures 177, each including three post pre-formed apertures 177 which are axially spaced from each other, it is to be understood that any other arrangement and number of post pre-formed apertures 177 is contemplated.

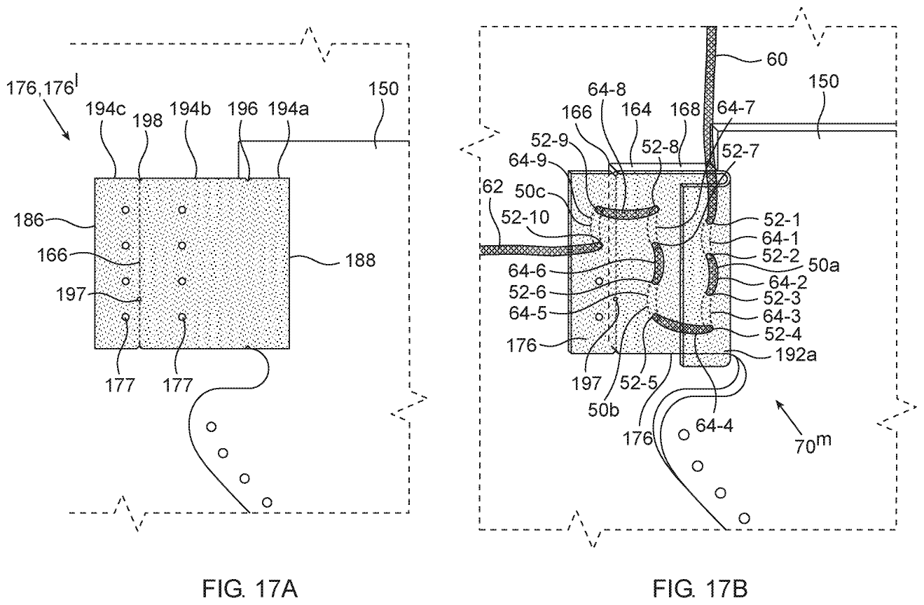

In some examples, any of the inner notches 196 and/or the outer notches 198 can be replaced by, or used in combination with, alternative modes of marking these positions along the reinforcement post 176, such as by ink-printed markers, marking apertures, and the like. FIG. 17A shows an exemplary reinforcement post 176^{l} placed on a leaflet 150 prior to forming a securement stitching pattern. Reinforcement post 176^{l} is an exemplary implementation of reinforcement post 176, and thus can include any of the features described for reinforcement post 176 throughout the current disclosure, except that the reinforcement post 176^{l} can further include one or more marking aperture 197 that can be used in a similar manner to that described above with respect to outer notches 198, to mark the desired position of the tab outer edge 166. Thus, as shown in FIG. 17A, the marking aperture 197 can assist in proper positioning of the reinforcement post 176^{l} relative to the tab 164 by aligning the marking aperture 197 with the tab outer edge 166.

While a marking aperture 197 is shown in FIG. 17A in combination with outer notches 198, it is to be understood that reinforcement post 176^{l} can be, in some examples, devoid of outer notches 198, and that marking aperture 197 can be solely used instead of outer notches 198. While a single marking aperture 197 is illustrated in FIG. 17A, it is to be understood that more than one apertures 197, such as two or more marking apertures 197 that can be axially spaced from each other, can be formed in a reinforcement post 176^{l}. Furthermore, it is to be understood that one or more marking apertures, similar to marking aperture 197, can be used in addition to, or instead of, inner notches 196, by being formed at a position of a first fold line 190a along which the reinforcement post 176 can be overfolded to form a first folded portion 192a.

FIG. 17B shows an exemplary securement stitching pattern 70^{m} used to secure a reinforcement post 176 to a tab 164. It is to be understood that the reinforcement post 176 is shown in FIG. 17B to include outer notches 198 and a marking aperture 197 by way of illustration and not limitation, and that any other type of reinforcement post 176 described herein can be secured to a tab 164 by a securement stitching pattern 70^{m}.

An inner fold vertical stitch line 50a of a securement stitching pattern 70^{m} includes a first vertical stitch 64-1 extending over an inner surface of the first folded portion 192a between a first penetration point 52-1 and a second penetration point 52-2, a second vertical stitch 64-2 extending over an outer surface of the first folded portion 192a between the second penetration point 52-2 and a third penetration point 52-3, and a third vertical stitch 64-3 extending over the inner surface of the first folded portion 192a between the third penetration point 52-3 and a fourth penetration point 52-4. A tail 62 of the suture 60 can extend from the first penetration point 52-1, for example along the outer surface of the folded portion 192a. The first penetration point 52-1 is distally offset from the post outflow edge 182, the second penetration point 52-2 is distal to the first penetration point 52-1, the third penetration point 52-3 is distal to the second penetration point 52-2, and the fourth penetration point 52-4 is distal to the third penetration point 52-3 and is proximally offset from the post inflow edge 184.

The securement stitching pattern 70^{m} further includes a fourth stitch 64-4, which is a horizontal stitch, extending between the fourth penetration point 52-4 of the inner fold vertical stitch line 50a and a fifth penetration point 52-5 of the post securement vertical stitch line 50b. The horizontal stitch 64-4 is disposed over outer surfaces of the reinforcement post 176, extending over part of the folded portion 192a, the post inner edge 188, and part of the second post section 194b.

The post securement vertical stitch line 50b of the securement stitching pattern 70^{m} includes a fifth vertical stitch 64-5 extending between the fifth penetration point 52-5 and a sixth penetration point 52-6 along the inner surface 160 of the tab 164, a sixth vertical stitch 64-6 extending between the sixth penetration point 52-6 and a seventh penetration point 52-7 along the first side 178 of the reinforcement post 176, and a seventh vertical stitch 64-7 extending between the seventh penetration point 52-7 and an eight penetration point 52-8 along the inner surface 160 of the tab 164. The eight penetration point 52-8 is distally offset from the tab outflow edge 168, the seventh penetration point 52-7 is distal to the sixth penetration point 52-6, the sixth penetration point 52-6 is distal to the seventh penetration point 52-7, and the fifth sixth penetration point 52-5 is distal to the sixth penetration point 52-6 and is proximally offset from the tab inflow edge 170.

The securement stitching pattern 70^{m} further includes an eight stitch 64-8, which is a horizontal stitch, extending between the eight penetration point 52-8 of the post securement vertical stitch line 50b and a ninth penetration point 52-9 of a third vertical stitch line 50c along a yet unfolded third post section 194c. The horizontal stitch 64-8 is disposed over the first side 178 of the reinforcement post 176, crossing the position of the tab outer edge 166.

The third vertical stitch line 50c of the securement stitching pattern 70^{m} includes a ninth vertical stitch 64-9 extending between the ninth penetration point 52-9 and a tenth penetration point 52-10 along second side 180 of the reinforcement post 176. The ninth penetration point 52-9 is distally offset from the post outflow edge 182, and the tenth penetration point 52-10 is distal to the ninth penetration point 52-9 and is proximally offset from the post inflow edge 184. A tail 62 of the suture 60 can extend from the tenth penetration point 52-10, for example along the first side of the reinforcement post 176.

In some examples, after passing the tab 164 through the window opening 118, a second folded portion 192b is formed and additional stitches and/or loops are added, as part of a complementary stitching pattern, along the third vertical stitch line 50c, to transform the third vertical stitch line to an outer fold vertical stitch line 50c.

As shown in FIG. 17B, the inner fold vertical stitch line 50a of securement stitching pattern 70^{m} includes three subsequent vertical stitches 64, wherein the uppermost vertical stitch 64-1 is distally offset from the post outflow edge 182, and the lowermost vertical stitch 64-3 is proximally offset from the post inflow edge 184. The post securement vertical stitch line 50b of securement stitching pattern 70^{m} includes three subsequent vertical stitches 64, wherein the uppermost vertical stitch 64-7 is distally offset from the tab outflow edge 168, and wherein the lowermost vertical stitch 64-5 is proximally offset from the tab inflow edge 170. The third vertical stitch line 50c of securement stitching pattern 70^{m} includes a single vertical stitch 64-9 which is axially offset from both the post outflow edge 182 and the post inflow edge 184, and can be closer to the post outflow edge 182 than to the post inflow edge 184.

The securement stitching pattern 70^{g} further includes a single horizontal stitch 64-4 extending between the inner fold vertical stitch line 50a and the post securement vertical stitch line 50b, and a single horizontal stitch 64-8 extending from the post securement vertical stitch line 50b and the third vertical stitch line 50c.

### Some Examples of the Disclosed Implementations

Some examples of above-described implementations are enumerated below. It should be noted that one feature of an example in isolation or more than one feature of the example taken in combination and, optionally, in combination with one or more features of one or more examples below are examples also falling within the disclosure of this application.

Example 1. A method for forming a commissure of a prosthetic valve, comprising:
attaching a reinforcement post to a tab of each leaflet of a plurality of leaflets, by threading a suture through the reinforcement post and the tab along a post securement vertical stitch line, wherein the post securement vertical stitch line does not extend along an entire height of the tab;
extending two tabs, one from each of two adjacent leaflets of the plurality of leaflets, through a window opening of a commissure support of a frame of a prosthetic valve, such that the post securement vertical stitch lines are positioned radially outwards from the commissure support; and
attaching the two tabs of the two leaflets to sidewalls of the commissure support.

Example 2. The method of any example herein, particularly of example, wherein the reinforcement post is positioned over an outer surface of the leaflet it is attached to, such that after the extending the two tabs through the window opening, the reinforcement posts attached to the two tabs of the two adjacent leaflets are separated from each other by the two tabs.

Example 3. The method of any example herein, particularly of any one of examples 1 or 2, further comprising, prior to the extending the two tabs through the window opening, forming a first folded portion of the reinforcement post by folding an inner edge of the reinforcement post outwards.

Example 4. The method of any example herein, particularly of example 3, further comprising threading the suture through the first folded portion along an inner fold vertical stitch line.

Example 5. The method of any example herein, particularly of example 4, wherein the extending the two tabs through the window opening further comprises positioning the inner fold vertical stitch line radially inwards to the commissure support.

Example 6. The method of any example herein, particularly of any one of examples 3 or 4, wherein the extending the two tabs through the window opening further comprises positioning the first folded portion radially inwards to the commissure support.

Example 7. The method of any example herein, particularly of any one of examples 4 to 6, further comprising, prior to the extending the two tabs through the window opening, forming at least one horizontal stitch extending between the inner fold vertical stitch line and the post securement vertical stitch line.

Example 8. The method of any example herein, particularly of example 7, wherein the at least one horizontal stitch extends over the inner edge of the reinforcement post.

Example 9. The method of any example herein, particularly of any one of examples 7 or 8, wherein, after the extending the two tabs through the window opening, the at least one horizontal stitch extends through the window opening.

Example 10. The method of any example herein, particularly of any one of examples 7 to 9, wherein the inner fold vertical stitch line, the post securement vertical stitch line, and the at least one horizontal stitch, are continuously formed by a single suture.

Example 11. The method of any example herein, particularly of any one of examples 7 to 10, wherein the inner fold vertical stitch line does not extend through the leaflet.

Example 12. The method of any example herein, particularly of any one of examples 7 to 11, wherein the post securement vertical stitch line comprises two vertical stitches.

Example 13. The method of any example herein, particularly of any one of examples 12, wherein the two vertical stitches of the post securement vertical stitch line comprise an uppermost vertical stitch which is distally offset from an outflow edge of the tab, and a lowermost vertical stitch which is proximally offset from an inflow edge of the tab.

Example 14. The method of any example herein, particularly of example 13, wherein the uppermost vertical stitch of the post securement vertical stitch line is disposed over a first side of the reinforcement post, and wherein the lowermost vertical stitch of the post securement vertical stitch line is disposed over an inner surface of the tab.

Example 15. The method of any example herein, particularly of example 14, wherein a tail of the suture extends from an uppermost penetration point of the uppermost vertical stitch of the post securement vertical stitch line, at least partially along the inner surface of the tab.

Example 16. The method of any example herein, particularly of example 14, further comprising a diagonal stitch extending from an uppermost penetration point of the uppermost vertical stitch of the post securement vertical stitch line, in a direction oriented distally and towards an outer edge of the reinforcement post.

Example 17. The method of any example herein, particularly of example 16, wherein the diagonal stitch extends partially over the inner surface of the tabs and past an outer edge of the tab.

Example 18. The method of any example herein, particularly of example 17, wherein a tail of the suture extends from the diagonal stitch, at least partially along a second side of the reinforcement post.

Example 19. The method of any example herein, particularly of any one of examples 12to 18, wherein the inner fold vertical stitch line comprises two vertical stitches.

Example 20. The method of any example herein, particularly of example 19, wherein the two vertical stitches of the inner fold vertical stitch line comprise an uppermost vertical stitch which is distally offset from an outflow edge of the reinforcement post, and a lowermost vertical stitch which is proximally offset from an inflow edge of the reinforcement post.

Example 21. The method of any example herein, particularly of example 20, wherein the uppermost vertical stitch of the inner fold vertical stitch line is disposed over an outer surface of the first folded portion, and wherein the lowermost vertical stitch of the inner fold vertical stitch line is disposed over an inner surface of the first folded portion.

Example 22. The method of any example herein, particularly of any one of examples 12 to 15, wherein the inner fold vertical stitch line comprises three vertical stitches.

Example 23. The method of any example herein, particularly of example 22, wherein the two vertical stitches of the inner fold vertical stitch line comprise a first vertical stitch which is distally offset from an outflow edge of the reinforcement post, a second vertical stitch which is distal to the first vertical stitch, and a third vertical stitch which is distal to the second vertical stitch and is proximally offset from an inflow edge of the reinforcement post.

Example 24. The method of any example herein, particularly of example 23, wherein the second vertical stitch of the inner fold vertical stitch line is disposed over an outer surface of the first folded portion, and wherein both the first vertical stitch and the third vertical stitch of the inner fold vertical stitch line are disposed over an inner surface of the first folded portion.

Example 25. The method of any example herein, particularly of any one of examples 12 to 15, wherein the inner fold vertical stitch line comprises a single vertical stitch.

Example 26. The method of any example herein, particularly of any one of examples 25, wherein the vertical stitch of the inner fold vertical stitch line is axially offset from an outflow edge and an inflow edge of the reinforcement post.

Example 27. The method of any example herein, particularly of example 26, wherein the vertical stitch of the inner fold vertical stitch line is disposed over an inner surface of the first folded portion.

Example 28. The method of any example herein, particularly of any one of examples 26 or 27, wherein the vertical stitch of the inner fold vertical stitch line is closer to the inflow edge of the reinforcement post than to the outflow edge of the reinforcement post.

Example 29. The method of any example herein, particularly of any one of examples 12 to 15, further comprising, after the extending the two tabs through the window opening, forming a second folded portion of the reinforcement post positioned radially outwards to the commissure support.

Example 30. The method of any example herein, particularly of example 29, further comprising threading the suture through the second folded portion along an outer fold vertical stitch line.

Example 31. The method of any example herein, particularly of example 30, wherein the outer fold vertical stitch line comprises an uppermost loop extending around an outflow edge of the reinforcement post, and a lowermost loop extending around an inflow edge of the reinforcement post.

Example 32. The method of any example herein, particularly of example 31, wherein the outer fold vertical stitch line further comprises an uppermost vertical stitch which is distal to the uppermost loop, and two vertical stitches extending between the uppermost vertical stitch and the lowermost loop, wherein the two vertical stitches extend between mutual penetration points, each over an opposite surface of the second folded portion.

Example 33. The method of any example herein, particularly of examples 7 to 11, wherein the post securement vertical stitch line comprises an uppermost loop distally offset from an outflow edge of the tab, a lowermost loop extending around an inflow edge of the tab, and a vertical stitch extending between the uppermost loop and the lowermost loop.

Example 34. The method of any example herein, particularly of example 33, wherein the vertical stitch of the inner fold vertical stitch line is disposed over an inner surface of the tab.

Example 35. The method of any example herein, particularly of any one of examples 33 or 34, wherein a tail of the suture extends from a penetration point shared by the lowermost loop and the vertical stitch of the post securement vertical stitch line, and extends at least partially over a first side of the reinforcement post.

Example 36. The method of any example herein, particularly of any one of examples 33 to 35, wherein the inner fold vertical stitch line comprises a single vertical stitch.

Example 37. The method of any example herein, particularly of example 36, wherein the vertical stitch of the inner fold vertical stitch line is axially offset from an outflow edge and an inflow edge of the reinforcement post.

Example 38. The method of any example herein, particularly of example 37, wherein the vertical stitch of the inner fold vertical stitch line is disposed over an inner surface of the first folded portion.

Example 39. The method of any example herein, particularly of any one of examples 37 or 38, wherein the vertical stitch of the inner fold vertical stitch line is closer to the outflow edge of the reinforcement post than to the inflow edge of the reinforcement post.

Example 40. The method of any example herein, particularly of any one of examples 7 to 11, wherein the post securement vertical stitch line comprises a loop distally offset from an outflow edge of the tab, and a vertical stitch distal to the loop and proximally offset from an inflow edge of the tab.

Example 41. The method of any example herein, particularly of example 40, wherein the vertical stitch of the inner fold vertical stitch line is disposed over an inner surface of the tab.

Example 42. The method of any example herein, particularly of any one of examples 40 or 41, wherein a tail of the suture extends from a penetration point shared by the loop and the vertical stitch, and extends at least partially over a first side of the reinforcement post.

Example 43. The method of any example herein, particularly of any one of examples 40 to 42, wherein the inner fold vertical stitch line comprises two vertical stitches.

Example 44. The method of any example herein, particularly of example 43, wherein the two vertical stitches of the inner fold vertical stitch line comprise an uppermost vertical stitch which is distally offset from an outflow edge of the reinforcement post, and a lowermost vertical stitch which is proximally offset from the inflow edge of the reinforcement post.

Example 45. The method of any example herein, particularly of examples 44, wherein the uppermost vertical stitch of the inner fold vertical stitch line is disposed over an outer surface of the first folded portion, and wherein the lowermost vertical stitch of the inner fold vertical stitch line is disposed over an inner surface of the first folded portion.

Example 46. The method of any example herein, particularly of any one of examples 7 to 11, further comprising, prior to the extending the two tabs through the window opening, threading the suture through a section of the reinforcement post extending past an outer edge of the tab, along a third vertical stitch line.

Example 47. The method of any example herein, particularly of example 46, wherein, after the extending the two tabs through the window opening, the third vertical stitch line is positioned radially outwards to the commissure support.

Example 48. The method of any example herein, particularly of any one of examples 46 or 47, further comprising, prior to the extending the two tabs through the window opening, maintaining the section of the reinforcement post along which the third vertical stitch line is formed in an unfolded configuration.

Example 49. The method of any example herein, particularly of any one of examples 46 to 48, wherein the post securement vertical stitch line comprises an uppermost vertical stitch distally offset from an outflow edge of the tab, a lowermost vertical stitch proximally offset from an inflow edge of the tab, and an intermediate vertical stitch extending between the uppermost vertical stitch and the lowermost vertical stitch.

Example 50. The method of any example herein, particularly of example 49, wherein the uppermost and the lowermost vertical stitches of the post securement vertical are disposed over an inner surface of the tab, and wherein the intermediate vertical stitch is disposed over a first side of the reinforcement post.

Example 51. The method of any example herein, particularly of any one of examples 46 to 50, wherein the third vertical stitch line comprises a single vertical stitch.

Example 52. The method of any example herein, particularly of example 51, wherein the vertical stitch of the third vertical stitch line is axially offset from an outflow edge and an inflow edge of the reinforcement post.

Example 53. The method of any example herein, particularly of example 52, wherein the vertical stitch of the third vertical stitch line is disposed over a second side of the reinforcement post.

Example 54. The method of claim 52 or 53, wherein the vertical stitch of the third vertical stitch line is closer to the outflow edge of the reinforcement post than to the inflow edge of the reinforcement post.

Example 55. The method of any example herein, particularly of any one of examples 52 to 55, wherein a tail of the suture extends from a penetration point of the vertical stitch of the third vertical stitch line.

Example 56. The method of any example herein, particularly of any one of examples 52 to 55, wherein the inner fold vertical stitch line comprises an uppermost vertical stitch distally offset from the outflow edge of the reinforcement post, a lowermost vertical stitch proximally offset from the inflow edge of the reinforcement post, and an intermediate vertical stitch extending between the uppermost vertical stitch and the lowermost vertical stitch.

Example 57. The method of any example herein, particularly of example 56, wherein the uppermost and the lowermost vertical stitches of the post securement vertical stitch line are disposed over an inner surface of the first folded portion, and wherein the intermediate vertical stitch is disposed over an outer surface of the first folded portion.

Example 58. The method of any example herein, particularly of any one of examples 7 to 11, wherein the post securement vertical stitch line comprises a loop extending around an inflow edge of the tab, an uppermost vertical stitch distally offset from an outflow edge of the tab, and two vertical stitches extending between the uppermost vertical stitch and the loop, wherein the two vertical stitches extend between mutual penetration points.

Example 59. The method of any example herein, particularly of example 58, wherein one of the two vertical stitches of the post securement vertical stitch line extends over a first side of the reinforcement post, and the other one of the two vertical stitches extends over an inner surface of the tab.

Example 60. The method of any example herein, particularly of example 59, wherein the uppermost vertical stitch of the post securement vertical stitch line extends over the inner surface of the tab.

Example 61. The method of any example herein, particularly of any one of examples 58 to 60, wherein a tail of the suture extends from an uppermost penetration point of the post securement vertical stitch line.

Example 62. The method of any example herein, particularly of any one of examples 58 to 61, wherein the inner fold vertical stitch line comprises a single vertical stitch.

Example 63. The method of any example herein, particularly of example 62, wherein the vertical stitch of the inner fold vertical stitch line is axially offset from an outflow edge and an inflow edge of the reinforcement post.

Example 64. The method of any example herein, particularly of example 63, wherein the vertical stitch of the inner fold vertical stitch line is disposed over an inner surface of the first folded portion.

Example 65. The method of any example herein, particularly of any one of examples 63 or 64, wherein the vertical stitch of the inner fold vertical stitch line is closer to the outflow edge of the reinforcement post than to the inflow edge of the reinforcement post.

Example 66. The method of any example herein, particularly of any one of examples 63 to 65, wherein further comprising, after the extending the two tabs through the window opening, forming a second folded portion of the reinforcement post positioned radially outwards to the commissure support.

Example 67. The method of any example herein, particularly of example 66, further comprising threading the suture through the second folded portion along an outer fold vertical stitch line.

Example 68. The method of any example herein, particularly of example 67, wherein the outer fold vertical stitch line comprises a single vertical suture axially offset from the outflow edge and the inflow edge of the reinforcement post.

Example 69. The method of any example herein, particularly of example 68, wherein the vertical stitch of the outer fold vertical stitch line is disposed over an outer surface of the second folded portion.

Example 70. The method of any example herein, particularly of any one of examples 68 or 69, wherein the vertical stitch of the outer fold vertical stitch line is closer to the outflow edge of the reinforcement post than to the inflow edge of the reinforcement post.

Example 71. The method of any example herein, particularly of any one of examples 68 to 70, wherein a tail of the suture extends from a penetration point aligned with, and distal to, the vertical stitch of the outer fold vertical stitch line.

Example 72. The method of any example herein, particularly of any one of examples 67 to 71, further comprising three horizontal stitches extending between the post securement vertical stitch line and the outer fold vertical stitch line, axially spaced from each other.

Example 73The method of any one of claims 12 to 72, wherein the at least one horizontal stitch extending between the inner fold vertical stitch line and the post securement vertical stitch line comprises a single horizontal stitch.

Example 74. The method of any example herein, particularly of any one of examples 7 to 11, wherein the post securement vertical stitch line comprises a loop extending around an inflow edge of the tab, an uppermost vertical stitch distally offset from an outflow edge of the tab, and lowermost vertical stitch extending between the uppermost vertical stitch and the loop.

Example 75. The method of any example herein, particularly of example 74, wherein the uppermost and the lowermost vertical stitches of the post securement vertical stitch line extend over an inner surface of the tab.

Example 76. The method of any example herein, particularly of any one of examples 74 or 75, wherein a tail of the suture extends from a penetration point shared by the uppermost and the lowermost vertical stitches of the post securement vertical stitch line.

Example 77. The method of any example herein, particularly of any one of examples 74 to 76, wherein the inner fold vertical stitch line comprises a single vertical stitch.

Example 78. The method of any example herein, particularly of examples 77, wherein the vertical stitch of the inner fold vertical stitch line is axially offset from an outflow edge and an inflow edge of the reinforcement post.

Example 79. The method of any example herein, particularly of example 78, wherein the vertical stitch of the inner fold vertical stitch line is disposed over an inner surface of the first folded portion.

Example 80. The method of any example herein, particularly of any one of examples 77 or 78, wherein the vertical stitch of the inner fold vertical stitch line is closer to the inflow edge of the reinforcement post than to the outflow edge of the reinforcement post.

Example 81. The method of any example herein, particularly of any one of examples 74 to 80, wherein the at least one horizontal stitch extending between the inner fold vertical stitch line and the post securement vertical stitch line comprises two horizontal stitches axially spaced from each other.

Example 82. The method of any example herein, particularly of any one of examples 3 to 81, wherein the forming the first folded portion comprises folding at a position of one or more inner notches of the reinforcement tab.

Example 83. The method of any example herein, particularly of any one of examples 1 or 2, wherein the post securement vertical stitch line comprises an uppermost loop distally offset from an outflow edge of the tab, a lowermost loop extending around an inflow edge of the tab, and a vertical stitch extending between the uppermost loop and the lowermost loop.

Example 84. The method of any example herein, particularly of example 83, wherein the vertical stitch of the post securement vertical stitch line is disposed over an inner surface of the tab.

Example 85. The method of any example herein, particularly of any one of examples 83 or 84, wherein, prior to the extending the two tabs through the window opening, the reinforcement post does is not folded along a portion thereof configured to be positioned radially inwards to the commissure support after the extending the two tabs through the window opening.

Example 86. The method of any example herein, particularly of any one of examples 1 to 85, wherein the tab comprises a plurality of tab pre-formed apertures.

Example 87. The method of any example herein, particularly of any one of examples 1 to 86, wherein the reinforcement post comprises a plurality of post pre-formed apertures.

Example 88. The method of any example herein, particularly of any one of examples 1 to 87, further comprising, prior to the attaching, placing the reinforcement post over the leaflet such that one or more outer notches of the reinforcement post are aligned with an outer edge of the tab.

Example 89. The method of any example herein, particularly of any one of examples 1 to 87, further comprising, prior to the attaching, placing the reinforcement post over the leaflet such that a marking aperture of the reinforcement post is aligned with an outer edge of the tab.

Example 90. The method of any example herein, particularly of any one of examples 1 to 89, further comprising, after the extending the two tabs through the window opening, placing a wedge member against the two tabs, radially outwards to the commissure support.

Example 91. The method of any example herein, particularly of any one of examples 1 to 90, wherein no suture line attaches any of the two leaflets to itself or to each other.

Example 92. The method of any example herein, particularly of any one of examples 1 to 91, wherein, after the extending the two tabs through the window opening, the reinforcement posts separate between the leaflets and the sidewalls of the commissure support.

Example 93. A method for forming a commissure of a prosthetic valve, comprising:
for each reinforcement post of a plurality of reinforcement posts, forming a first folded portion of the reinforcement post by folding an inner edge of the reinforcement post;
threading a suture through the first folded portion along an inner fold vertical stitch line;
attaching one of the plurality of reinforcement posts to a tab of each leaflet of a plurality of leaflets, by forming a horizontal stitch and threading the suture through the reinforcement post and the tab along a post securement vertical stitch line, wherein the horizontal stitch extends between the inner fold vertical stitch line and the post securement vertical stitch line;
extending two tabs, one from each of two adjacent leaflets of the plurality of leaflets, through a window opening of a commissure support of a frame of a prosthetic valve, such that the post securement vertical stitch lines are positioned radially outwards from the commissure support, and such that the inner fold vertical stitch lines are positioned radially inwards to the commissure support; and
attaching the two tabs of the two leaflets to sidewalls of the commissure support.

Example 94. The method of any example herein, particularly of example 93, wherein the reinforcement post is positioned over an outer surface of the leaflet it is attached to, such that after the extending the two tabs through the window opening, the reinforcement posts attached to the two tabs of the two adjacent leaflets are separated from each other by the two tabs.

Example 95. The method of any example herein, particularly of any one of examples 93 or 94, wherein the extending the two tabs through the window opening further comprises positioning the first folded portion radially inwards to the commissure support.

Example 96. The method of any example herein, particularly of any one of examples 93 to 95, wherein the horizontal stitch extends over an inner edge of the reinforcement post.

Example 97. The method of any example herein, particularly of any one of examples 93 to 96, wherein, after the extending the two tabs through the window opening, the horizontal stitch extends through the window opening.

Example 98. The method of any example herein, particularly of any one of examples 93 to 97, wherein the inner fold vertical stitch line does not extend through the leaflet.

Example 99. The method of any example herein, particularly of any one of examples 93 to 98, wherein the post securement vertical stitch line comprises a lowermost loop extending around an inflow edge of the tab, and an uppermost loop extending around an outflow edge of the tab.

Example 100. The method of any example herein, particularly of example 99, wherein the post securement vertical stitch line further comprises a lowermost vertical stitch proximal to the lowermost loop, and two vertical stitches extending between the lowermost vertical stitch and the uppermost loop, wherein the two vertical stitches extend between mutual penetration points.

Example 101. The method of any example herein, particularly of example 100, wherein one of the two vertical stitches of the post securement vertical stitch line extends over a first side of the reinforcement post, and the other one of the two vertical stitches extends over an inner surface of the tab.

Example 102. The method of any example herein, particularly of example 101, wherein the lowermost vertical stitch of the post securement vertical stitch line extends over the inner surface of the tab.

Example 103. The method of any one of claims 99 to 102, further comprising forming a horizontal stitch extending from the lowermost loop of the post securement vertical stitch line to a penetration point formed at a section of the reinforcement post extending past an outer edge of the tab.

Example 104. The method of any example herein, particularly of any one of examples 99 to 104, wherein the inner fold vertical stitch line comprises a single vertical stitch.

Example 105. The method of any example herein, particularly of example 104, wherein the vertical stitch of the inner fold vertical stitch line is axially offset from an outflow edge and an inflow edge of the reinforcement post.

Example 106. The method of any example herein, particularly of example 105, wherein the vertical stitch of the inner fold vertical stitch line is disposed over an inner surface of the first folded portion.

Example 107. The method of any example herein, particularly of any one of examples 105 or 106, wherein the vertical stitch of the inner fold vertical stitch line is closer to the inflow edge of the reinforcement post than to the outflow edge of the reinforcement post.

Example 108. The method of any example herein, particularly of any one of examples 93 to 107, wherein the forming the first folded portion comprises folding at a position of one or more inner notches of the reinforcement tab.

Example 109. The method of any one of claims 93 to 108, further comprising, prior to the attaching, placing the reinforcement post over the leaflet such that one or more outer notches of the reinforcement post are aligned with an outer edge of the tab.

Example 110. The method of any example herein, particularly of any one of examples 93 to 109, further comprising, prior to the attaching, placing the reinforcement post over the leaflet such that marking aperture of the reinforcement post is aligned with an outer edge of the tab.

Example 111. A prosthetic valve comprising:
a frame comprising a plurality of commissure supports, each commissure support comprising two sidewalls defining a window opening therebetween;
a plurality of leaflets, each leaflet comprising opposing tabs disposed on opposite sides of the leaflet; and
a plurality of commissures secured to the plurality of commissure supports, each commissure comprising two tabs, one from each of two adjacent leaflets of the plurality of leaflets, and two reinforcement posts, each one of the reinforcement posts attached to a corresponding one of the two tabs of the commissure by a securement stitching pattern which comprises a post securement vertical stitch line positioned radially outwards from the commissure support;
wherein post securement vertical stitch line does not extend along an entire height of the tab.

Example 112. The prosthetic valve of any example herein, particularly of example 111, wherein the securement stitching pattern is formed off the frame.

Example 113. The prosthetic valve of any example herein, particularly of any one of examples 111 or 112, wherein each reinforcement post is positioned on an outer surface of the leaflet.

Example 114. The prosthetic valve of any example herein, particularly of any one of examples 111 to 113, wherein each securement stitching pattern is formed by a single suture.

Example 115. The prosthetic valve of any example herein, particularly of any one of examples 111 to 114, wherein each reinforcement post comprises a first folded portion, and wherein the securement stitching pattern further comprises an inner fold vertical stitch line radially inwards to the commissure support, extending through the first folded portion, and at least one horizontal stitch extending between the inner fold vertical stitch line and the post securement vertical stitch line.

Example 116. The prosthetic valve of any example herein, particularly of example 115, wherein the inner fold vertical stitch line does not extend through the leaflet.

Example 117. The prosthetic valve of any example herein, particularly of any one of examples 115 or 116, wherein the at least one horizontal stitch extends over an inner edge of the reinforcement post.

Example 118. The prosthetic valve of any example herein, particularly of any one of examples 115 to 117, wherein the at least one horizontal stitch extends through the window opening.

Example 119. The prosthetic valve of any example herein, particularly of any one of examples 115 to 118, wherein the post securement vertical stitch line comprises two vertical stitches.

Example 120. The prosthetic valve of any example herein, particularly of example 119, wherein the two vertical stitches of the post securement vertical stitch line comprise an uppermost vertical stitch which is distally offset from an outflow edge of the tab, and a lowermost vertical stitch which is proximally offset from an inflow edge of the tab.

Example 121. The prosthetic valve of any example herein, particularly of example 120, wherein the uppermost vertical stitch of the post securement vertical stitch line is disposed over a first side of the reinforcement post, and wherein the lowermost vertical stitch of the post securement vertical stitch line is disposed over an inner surface of the tab.

Example 122. The prosthetic valve of any example herein, particularly of example 121, wherein the securement stitching pattern further comprises a diagonal stitch extending from an uppermost penetration point of the uppermost vertical stitch of the post securement vertical stitch line, in a direction oriented distally and towards an outer edge of the reinforcement post.

Example 123. The prosthetic valve of any example herein, particularly of example 122, wherein the diagonal stitch extends partially over the inner surface of the tabs and past an outer edge of the tab.

Example 124. The prosthetic valve of any example herein, particularly of any one of examples 119 to 123, wherein the inner fold vertical stitch line comprises two vertical stitches.

Example 125. The prosthetic valve of any example herein, particularly of example 124, wherein the two vertical stitches of the inner fold vertical stitch line comprise an uppermost vertical stitch which is distally offset from an outflow edge of the reinforcement post, and a lowermost vertical stitch which is proximally offset from an inflow edge of the reinforcement post.

Example 126. The prosthetic valve of any example herein, particularly of examples 125, wherein the uppermost vertical stitch of the inner fold vertical stitch line is disposed over an outer surface of the first folded portion, and wherein the lowermost vertical stitch of the inner fold vertical stitch line is disposed over an inner surface of the first folded portion.

Example 127. The prosthetic valve of any example herein, particularly of any one of examples 119 to 121, wherein the inner fold vertical stitch line comprises three vertical stitches.

Example 128. The prosthetic valve of any example herein, particularly of example 127, wherein the three vertical stitches of the inner fold vertical stitch line comprise a first vertical stitch which is distally offset from an outflow edge of the reinforcement post, a second vertical stitch which is distal to the first vertical stitch, and a third vertical stitch which is distal to the second vertical stitch and is proximally offset from an inflow edge of the reinforcement post.

Example 129. The prosthetic valve of any example herein, particularly of example 128, wherein the second vertical stitch of the inner fold vertical stitch line is disposed over an outer surface of the first folded portion, and wherein both the first vertical stitch and the third vertical stitch of the inner fold vertical stitch line are disposed over an inner surface of the first folded portion.

Example 130. The prosthetic valve of any example herein, particularly of any one of examples 119 to 121, wherein the inner fold vertical stitch line comprises a single vertical stitch.

Example 131. The prosthetic valve of any example herein, particularly of example 130, wherein the vertical stitch of the inner fold vertical stitch line is axially offset from an outflow edge and an inflow edge of the reinforcement post.

Example 132. The prosthetic valve of any example herein, particularly of example 131, wherein the vertical stitch of the inner fold vertical stitch line is disposed over an inner surface of the first folded portion.

Example 133. The prosthetic valve of any example herein, particularly of example 131 or 132, wherein the vertical stitch of the inner fold vertical stitch line is closer to the inflow edge of the reinforcement post than to the outflow edge of the reinforcement post.

Example 134. The prosthetic valve of any example herein, particularly of any one of examples 119 to 121, wherein each reinforcement post further comprises a second folded portion radially outwards to the commissure support.

Example 135. The prosthetic valve of any example herein, particularly of example 134, wherein each commissure further comprises a complementary stitching pattern which comprises an outer fold vertical stitch line threaded through the second folded portion along.

Example 136. The prosthetic valve of any example herein, particularly of example 135, wherein the outer fold vertical stitch line comprises an uppermost loop extending around an outflow edge of the reinforcement post, and a lowermost loop extending around an inflow edge of the reinforcement post.

Example 137. The prosthetic valve of any example herein, particularly of examples 136, wherein the outer fold vertical stitch line further comprises an uppermost vertical stitch which is distal to the uppermost loop, and two vertical stitches extending between the uppermost vertical stitch and the lowermost loop, wherein the two vertical stitches extend between mutual penetration points, each over an opposite surface of the second folded portion.

Example 138. The prosthetic valve of any example herein, particularly of any one of examples 115 to 118, wherein the post securement vertical stitch line comprises an uppermost loop distally offset from an outflow edge of the tab, a lowermost loop extending around an inflow edge of the tab, and a vertical stitch extending between the uppermost loop and the lowermost loop.

Example 139. The prosthetic valve of any example herein, particularly of example 138, wherein the vertical stitch of the inner fold vertical stitch line is disposed over an inner surface of the tab.

Example 140. The prosthetic valve of any example herein, particularly of any one of examples 138 or 139, wherein the reinforcement post comprises a section extending past an outer edge of the tab.

Example 141. The prosthetic valve of any example herein, particularly of any one of examples 138 to 140, wherein the inner fold vertical stitch line comprises a single vertical stitch.

Example 142. The prosthetic valve of any example herein, particularly of example 141, wherein the vertical stitch of the inner fold vertical stitch line is axially offset from an outflow edge and an inflow edge of the reinforcement post.

Example 143. The prosthetic valve of any example herein, particularly of example 142, wherein the vertical stitch of the inner fold vertical stitch line is disposed over an inner surface of the first folded portion.

Example 144. The prosthetic valve of any example herein, particularly of any one of examples 141 or 142, wherein the vertical stitch of the inner fold vertical stitch line is closer to the outflow edge of the reinforcement post than to the inflow edge of the reinforcement post.

Example 145. The prosthetic valve of any example herein, particularly of any one of examples 115 to 118, wherein the post securement vertical stitch line comprises a loop distally offset from an outflow edge of the tab, and a vertical stitch distal to the loop and proximally offset from an inflow edge of the tab.

Example 146. The prosthetic valve of any example herein, particularly of example 145, wherein the vertical stitch of the inner fold vertical stitch line is disposed over an inner surface of the tab.

Example 147. The prosthetic valve of any example herein, particularly of any one of examples 145 or 146, wherein the inner fold vertical stitch line comprises two vertical stitches.

Example 148. The prosthetic valve of claim 147, wherein the two vertical stitches of the inner fold vertical stitch line comprise an uppermost vertical stitch which is distally offset from an outflow edge of the reinforcement post, and a lowermost vertical stitch which is proximally offset from the inflow edge of the reinforcement post.

Example 149. The prosthetic valve of any example herein, particularly of example 148, wherein the uppermost vertical stitch of the inner fold vertical stitch line is disposed over an outer surface of the first folded portion, and wherein the lowermost vertical stitch of the inner fold vertical stitch line is disposed over an inner surface of the first folded portion.

Example 150. The prosthetic valve of any example herein, particularly of any one of examples 115 to 118, wherein the securement stitching pattern further comprises a third vertical stitch line extending through a section of the reinforcement post extending past an outer edge of the tab.

Example 151. The prosthetic valve of any example herein, particularly of example 150, wherein the third vertical stitch line is radially outwards to the commissure support.

Example 152. The prosthetic valve of any example herein, particularly of any one of examples 150 or 151, wherein the post securement vertical stitch line comprises an uppermost vertical stitch distally offset from an outflow edge of the tab, a lowermost vertical stitch proximally offset from an inflow edge of the tab, and an intermediate vertical stitch extending between the uppermost vertical stitch and the lowermost vertical stitch.

Example 153. The prosthetic valve of any example herein, particularly of example 152, wherein the uppermost and the lowermost vertical stitches of the post securement vertical are disposed over an inner surface of the tab, and wherein the intermediate vertical stitch is disposed over a first side of the reinforcement post.

Example 154. The prosthetic valve of any example herein, particularly of any one of examples 150 to 153, wherein the third vertical stitch line comprises a single vertical stitch.

Example 155. The prosthetic valve of any example herein, particularly of example 154, wherein the vertical stitch of the third vertical stitch line is axially offset from an outflow edge and an inflow edge of the reinforcement post.

Example 156. The prosthetic valve of any example herein, particularly of example 155, wherein the vertical stitch of the third vertical stitch line is disposed over a second side of the reinforcement post.

Example 157. The prosthetic valve of any example herein, particularly of example 154 or 155, wherein the vertical stitch of the third vertical stitch line is closer to the outflow edge of the reinforcement post than to the inflow edge of the reinforcement post.

Example 158. The prosthetic valve of any example herein, particularly of any one of examples 155 to 157, wherein the inner fold vertical stitch line comprises an uppermost vertical stitch distally offset from the outflow edge of the reinforcement post, a lowermost vertical stitch proximally offset from the inflow edge of the reinforcement post, and an intermediate vertical stitch extending between the uppermost vertical stitch and the lowermost vertical stitch.

Example 159. The prosthetic valve of any example herein, particularly of example 158, wherein the uppermost and the lowermost vertical stitches of the post securement vertical stitch line are disposed over an inner surface of the first folded portion, and wherein the intermediate vertical stitch is disposed over an outer surface of the first folded portion.

Example 160. The prosthetic valve of any example herein, particularly of any one of examples 115 to 118, wherein the post securement vertical stitch line comprises a loop extending around an inflow edge of the tab, an uppermost vertical stitch distally offset from an outflow edge of the tab, and two vertical stitches extending between the uppermost vertical stitch and the loop, wherein the two vertical stitches extend between mutual penetration points.

Example 161. The prosthetic valve of any example herein, particularly of example 160, wherein one of the two vertical stitches of the post securement vertical stitch line extends over a first side of the reinforcement post, and the other one of the two vertical stitches extends over an inner surface of the tab.

Example 162. The prosthetic valve of any example herein, particularly of example 161, wherein the uppermost vertical stitch of the post securement vertical stitch line extends over the inner surface of the tab.

Example 163. The prosthetic valve of any example herein, particularly of any one of examples 160 to 162, wherein the inner fold vertical stitch line comprises a single vertical stitch.

Example 164. The prosthetic valve of any example herein, particularly of example 163, wherein the vertical stitch of the inner fold vertical stitch line is axially offset from an outflow edge and an inflow edge of the reinforcement post.

Example 165. The prosthetic valve of any example herein, particularly of example 164, wherein the vertical stitch of the inner fold vertical stitch line is disposed over an inner surface of the first folded portion.

Example 166. The prosthetic valve of any example herein, particularly of example 163 or 164, wherein the vertical stitch of the inner fold vertical stitch line is closer to the outflow edge of the reinforcement post than to the inflow edge of the reinforcement post.

Example 167. The prosthetic valve of any example herein, particularly of any one of examples 164 to 166, wherein each reinforcement post further comprises a second folded portion radially outwards to the commissure support.

Example 168. The prosthetic valve of any example herein, particularly of example 167, wherein each commissure further comprises a complementary stitching pattern which comprises an outer fold vertical stitch line threaded through the second folded portion along.

Example 169. The prosthetic valve of any example herein, particularly of example 168, wherein the outer fold vertical stitch line comprises a single vertical suture axially offset from the outflow edge and the inflow edge of the reinforcement post.

Example 170. The prosthetic valve of any example herein, particularly of example 169, wherein the vertical stitch of the outer fold vertical stitch line is disposed over an outer surface of the second folded portion.

Example 171. The prosthetic valve of any example herein, particularly of example 169 or 170, wherein the vertical stitch of the outer fold vertical stitch line is closer to the outflow edge of the reinforcement post than to the inflow edge of the reinforcement post.

Example 172. The prosthetic valve of any example herein, particularly of any one of examples 168 to 171, further comprising three horizontal stitches extending between the post securement vertical stitch line and the outer fold vertical stitch line, axially spaced from each other.

Example 173. The prosthetic valve of any example herein, particularly of any one of examples 119 to 172, wherein the at least one horizontal stitch extending between the inner fold vertical stitch line and the post securement vertical stitch line comprises a single horizontal stitch.

Example 174. The prosthetic valve of any example herein, particularly of any one of examples 115 to 118, wherein the post securement vertical stitch line comprises a loop extending around an inflow edge of the tab, an uppermost vertical stitch distally offset from an outflow edge of the tab, and lowermost vertical stitch extending between the uppermost vertical stitch and the loop.

Example 175. The prosthetic valve of any example herein, particularly of example 174, wherein the uppermost and the lowermost vertical stitches of the post securement vertical stitch line extend over an inner surface of the tab.

Example 176. The prosthetic valve of claim 174 or 175, wherein the inner fold vertical stitch line comprises a single vertical stitch.

Example 177. The prosthetic valve of any example herein, particularly of example 176, wherein the vertical stitch of the inner fold vertical stitch line is axially offset from an outflow edge and an inflow edge of the reinforcement post.

Example 178. The prosthetic valve of any example herein, particularly of example 177, wherein the vertical stitch of the inner fold vertical stitch line is disposed over an inner surface of the first folded portion.

Example 179. The prosthetic valve of any example herein, particularly of any one of examples 177 or 178, wherein the vertical stitch of the inner fold vertical stitch line is closer to the inflow edge of the reinforcement post than to the outflow edge of the reinforcement post.

Example 180. The prosthetic valve of any example herein, particularly of any one of examples 174 to 179, wherein the at least one horizontal stitch extending between the inner fold vertical stitch line and the post securement vertical stitch line comprises two horizontal stitches axially spaced from each other.

Example 181. The prosthetic valve of any example herein, particularly of any one of examples 111 to 180, wherein each reinforcement post further comprises one or more inner notches at a first fold line of the first folded portion.

Example 182. The prosthetic valve of any example herein, particularly of any one of examples 111 to 181, wherein each reinforcement post further comprises one or more outer notches aligned with an outer edge of the tab.

Example 183. The prosthetic valve of any example herein, particularly of any one of examples 111 to 181, wherein each reinforcement post further comprises a marking aperture aligned with an outer edge of the tab.

Example 184. The prosthetic valve of any example herein, particularly of any one of examples 111 to 183, wherein no suture line attaches any of the two leaflets to itself or to each other.

Example 185. The prosthetic valve of any example herein, particularly of any one of examples 111 to 184, wherein the reinforcement posts of each commissure separate between the leaflets and the sidewalls of the commissure support.

Example 186: A prosthetic valve comprising:
a frame comprising a plurality of commissure supports, each commissure support comprising two sidewalls defining a window opening therebetween;
a plurality of leaflets, each leaflet comprising opposing tabs disposed on opposite sides of the leaflet; and
a plurality of commissures secured to the plurality of commissure supports, each commissure comprising two tabs, one from each of two adjacent leaflets of the plurality of leaflets, and two reinforcement posts, each one of the reinforcement posts attached to a corresponding one of the two tabs of the commissure by a securement stitching pattern which comprises a post securement vertical stitch line positioned radially outwards from the commissure support.

Example 187. The prosthetic valve of any example herein, particularly of example 186, wherein the securement stitching pattern is formed off the frame.

Example 188. The prosthetic valve of claim 186 or 187, wherein each stitching pattern is formed by a single suture.

It is appreciated that certain features of the disclosure, which are, for clarity, described in the context of separate examples, may also be provided in combination in a single example. Conversely, various features of the disclosure, which are, for brevity, described in the context of a single example, may also be provided separately or in any suitable sub-combination or as suitable in any other described example of the disclosure. No feature described in the context of an example is to be considered an essential feature of that example, unless explicitly specified as such.

In view of the many possible examples to which the principles of the disclosure may be applied, it should be recognized that the illustrated examples are only preferred examples and should not be taken as limiting the scope. Rather, the scope is defined by the following claims. We therefore claim all that comes within the scope and spirit of these claims.

1. A method for forming a commissure of a prosthetic valve, comprising:
attaching a reinforcement post to a tab of each leaflet of a plurality of leaflets, by threading a suture through the reinforcement post and the tab along a post securement vertical stitch line, wherein the post securement vertical stitch line does not extend along an entire height of the tab;
extending two tabs, one from each of two adjacent leaflets of the plurality of leaflets, through a window opening of a commissure support of a frame of a prosthetic valve, such that the post securement vertical stitch lines are positioned radially outwards from the commissure support; and
attaching the two tabs of the two leaflets to sidewalls of the commissure support.

2. The method of claim 1, further comprising, prior to the extending the two tabs through the window opening, forming a first folded portion of the reinforcement post by folding an inner edge of the reinforcement post outwards.

3. The method of claim 2, further comprising threading the suture through the first folded portion along an inner fold vertical stitch line.

4. The method of claim 3, wherein the extending the two tabs through the window opening further comprises positioning the inner fold vertical stitch line radially inwards to the commissure support.

5. The method of claim 3, further comprising, prior to the extending the two tabs through the window opening, forming at least one horizontal stitch extending between the inner fold vertical stitch line and the post securement vertical stitch line.

6. The method of claim 5, wherein the inner fold vertical stitch line, the post securement vertical stitch line, and the at least one horizontal stitch, are continuously formed by a single suture.

7. The method of claim 5, wherein the post securement vertical stitch line comprises two vertical stitches.

8. The method of claim 7, wherein the two vertical stitches of the post securement vertical stitch line comprise an uppermost vertical stitch which is distally offset from an outflow edge of the tab, and a lowermost vertical stitch which is proximally offset from an inflow edge of the tab.

9. The method of claim 8, wherein the uppermost vertical stitch of the post securement vertical stitch line is disposed over a first side of the reinforcement post, and wherein the lowermost vertical stitch of the post securement vertical stitch line is disposed over an inner surface of the tab.

10. The method of claim 9, wherein a tail of the suture extends from an uppermost penetration point of the uppermost vertical stitch of the post securement vertical stitch line, at least partially along the inner surface of the tab.

11. The method of claim 7, wherein the inner fold vertical stitch line comprises two vertical stitches.

12. The method of claim 11, wherein the two vertical stitches of the inner fold vertical stitch line comprise an uppermost vertical stitch which is distally offset from an outflow edge of the reinforcement post, and a lowermost vertical stitch which is proximally offset from an inflow edge of the reinforcement post.

13. The method of claim 12, wherein the uppermost vertical stitch of the inner fold vertical stitch line is disposed over an outer surface of the first folded portion, and wherein the lowermost vertical stitch of the inner fold vertical stitch line is disposed over an inner surface of the first folded portion.

14. The method of claim 7, further comprising, after the extending the two tabs through the window opening, forming a second folded portion of the reinforcement post positioned radially outwards to the commissure support.

15. The method of claim 14, further comprising threading the suture through the second folded portion along an outer fold vertical stitch line.

16. The method of claim 15, wherein the outer fold vertical stitch line comprises an uppermost loop extending around an outflow edge of the reinforcement post, and a lowermost loop extending around an inflow edge of the reinforcement post.

17. The method of claim 16, wherein the outer fold vertical stitch line further comprises an uppermost vertical stitch which is distal to the uppermost loop, and two vertical stitches extending between the uppermost vertical stitch and the lowermost loop, wherein the two vertical stitches extend between mutual penetration points, each over an opposite surface of the second folded portion.

18. A method for forming a commissure of a prosthetic valve, comprising:
for each reinforcement post of a plurality of reinforcement posts, forming a first folded portion of the reinforcement post by folding an inner edge of the reinforcement post;
threading a suture through the first folded portion along an inner fold vertical stitch line;
attaching one of the plurality of reinforcement posts to a tab of each leaflet of a plurality of leaflets, by forming a horizontal stitch and threading the suture through the reinforcement post and the tab along a post securement vertical stitch line, wherein the horizontal stitch extends between the inner fold vertical stitch line and the post securement vertical stitch line;
extending two tabs, one from each of two adjacent leaflets of the plurality of leaflets, through a window opening of a commissure support of a frame of a prosthetic valve, such that the post securement vertical stitch lines are positioned radially outwards from the commissure support, and such that the inner fold vertical stitch lines are positioned radially inwards to the commissure support; and
attaching the two tabs of the two leaflets to sidewalls of the commissure support.

19. The method of claim 18, wherein the inner fold vertical stitch line does not extend through the leaflet.

20. A prosthetic valve comprising:
a frame comprising a plurality of commissure supports, each commissure support comprising two sidewalls defining a window opening therebetween;
a plurality of leaflets, each leaflet comprising opposing tabs disposed on opposite sides of the leaflet; and
a plurality of commissures secured to the plurality of commissure supports, each commissure comprising two tabs, one from each of two adjacent leaflets of the plurality of leaflets, and two reinforcement posts, each one of the reinforcement posts attached to a corresponding one of the two tabs of the commissure by a securement stitching pattern which comprises a post securement vertical stitch line positioned radially outwards from the commissure support;
wherein post securement vertical stitch line does not extend along an entire height of the tab.

21. The prosthetic valve of claim 20, wherein the securement stitching pattern is formed off the frame.

22. The prosthetic valve of claim 20, wherein each securement stitching pattern is formed by a single suture.

23. The prosthetic valve of claim 20, wherein each reinforcement post comprises a first folded portion, and wherein the securement stitching pattern further comprises an inner fold vertical stitch line radially inwards to the commissure support, extending through the first folded portion, and at least one horizontal stitch extending between the inner fold vertical stitch line and the post securement vertical stitch line.

24. The prosthetic valve of claim 23, wherein the inner fold vertical stitch line does not extend through the leaflet.
